SEQUENCE LISTING (26 sequences)

SEQ ID NO: 1 - protein, 5 aa, Rattus norvegicus
SFPMA

SEQ ID NO: 2 - protein, 17 aa, Rattus norvegicus
TISTSGGRTYYRDSVKG

SEQ ID NO: 3 - protein, 10 aa, Rattus norvegicus
FRQYSGGFDY

SEQ ID NO: 4 - protein, 13 aa, Rattus norvegicus
TLSSGNIENNYVH

SEQ ID NO: 5 - protein, 7 aa, Rattus norvegicus
DDDKRPD

SEQ ID NO: 6 - protein, 9 aa, Rattus norvegicus
HSYVSSFNV

SEQ ID NO: 7 - protein, 30 aa, Rattus norvegicus
QVQLQESGGGLVQPGRSMKLSCAASGFTFS

SEQ ID NO: 8 - protein, 14 aa, Rattus norvegicus
WVRQAPKKGLEWVA

SEQ ID NO: 9 - protein, 32 aa, Rattus norvegicus
RFTISRDNGKSILYLQMNSLRSEDTATYYCSR

SEQ ID NO: 10 - protein, 10 aa, Rattus norvegicus
WGQGTTVTVS

SEQ ID NO: 11 - protein, 22 aa, Rattus norvegicus
DFMLTQPHSVSESPGKTVIISC

SEQ ID NO: 12 - protein, 15 aa, Rattus norvegicus
WYQQRPGRAPTLVIF

SEQ ID NO: 13 - protein, 34 aa, Rattus norvegicus
GVPDRFSGSIDRSSNSASLTISGLQTEDEADYYC

SEQ ID NO: 14 - protein, 10 aa, Rattus norvegicus
FGGGTKLTVL

SEQ ID NO: 15 - protein, 110 aa, Artificial Sequence
  Synthetically  generated peptide
DFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVL

SEQ ID NO: 16 - DNA, 330 bp, Artificial Sequence
  Synthetically  generated oligonucleotide
gactttatgcttactcagccccactctgtgtctgagtctcccggaaagacagtcattatttcttgcacactcagctctggtaacatagaaaacaactatgtgcactggtaccagcaaaggccgggaagagctcccaccctcgtgattttcgatgatgataagagaccggatggtgtccctgacaggttctctggctccattgacaggtcttccaactcagcctccctgacaatcagtggtctgcaaactgaagatgaagctgactactactgtcattcttatgttagtagttttaatgttttcggcggtggaacaaagctcactgtcctt

SEQ ID NO: 17 - protein, 119 aa, Rattus norvegicus
QVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSS

SEQ ID NO: 18 - DNA, 357 bp, Rattus norvegicus
  CDS (1)...(357)
caggtccaattgcaggagtctgggggcggtttagtgcagcctggaaggtccatgaaactctcctgtgcagcctcaggattcactttcagtagctttccaatggcctgggtccgccaggctccaaagaagggtctggagtgggtcgcaaccattagtactagtggtggtagaacttactatcgagactccgtgaagggccgattcactatctccagagataatgggaaaagcatcctatacctgcaaatgaatagtctgaggtctgaggacacggccacttattactgttcaagatttcggcagtacagtggtggctttgattactggggccaagggaccacggtcaccgtcagctca

SEQ ID NO: 19 - DNA, 15 bp, Rattus norvegicus
agctttccaatggcc

SEQ ID NO: 20 - DNA, 51 bp, Rattus norvegicus
accattagtactagtggtggtagaacttactatcgagactccgtgaagggc

SEQ ID NO: 21 - DNA, 30 bp, Rattus norvegicus
tttcggcagtacagtggtggctttgattac

SEQ ID NO: 22 - DNA, 39 bp, Rattus norvegicus
acactcagctctggtaacatagaaaacaactatgtgcac

SEQ ID NO: 23 - DNA, 21 bp, Rattus norvegicus
gatgatgataagagaccggat

SEQ ID NO: 24 - DNA, 27 bp, Rattus norvegicus
cattcttatgttagtagttttaatgtt

SEQ ID NO: 25 - protein, 310 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQKSLSLSPGK

SEQ ID NO: 26 - DNA, 993 bp, Homo sapiens
  CDS (1)...(990)
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2002/0147312 A1
(43) Pub. Date: Oct. 10, 2002

(54) Hybrid antibodies and uses thereof

(76) Inventors: Theresa O'Keefe, Waltham, MA (US); Patricia Rao, Acton, MA (US)

Correspondence Address: P. LOUIS MYERS, Fish & Richardson P.C., 225 Franklin Street, Boston, MA, 02110-2804, US

(21) Appl. No.: 10/060,714

(22) Filed: Jan. 30, 2002

Related U.S. Application Data
(60) Provisional application No. 60/265,914, filed on Feb. 2, 2001.

(51) Int. Cl. C07K 16/28

(52) U.S. Cl. 530/387.3; 530/388.15

(57) ABSTRACT

Methods of providing hybrid antibodies having improved assembly characteristics are disclosed. Accordingly, the present invention provides hybrid antibodies, antigen binding fragments thereof, which include a chimeric immunoglobulin heavy chain and a humanized or CDR-grafted immunoglobulin light chain, pharmaceutical compositions thereof, as well as nucleic acids encoding the aforesaid antibodies, host cells and vectors containing such nucleic acids. Therapeutic and diagnostic uses of such hybrid antibodies molecules thereof are also disclosed.

### RELATED APPLICATIONS

This application claims priority to U.S. provisional application number 60/265,914 filed on Feb. 2, 2001, the contents of which are incorporated herein by reference.

### BACKGROUND OF THE INVENTION

This invention relates to hybrid antibodies, or antigen-binding fragments thereof, having improved assembly characteristics and methods of making and using the same. The hybrid antibodies, or antigen-binding fragments thereof, of the invention include a chimeric immunoglobulin heavy chain and a humanized, or CDR-grafted, immunoglobulin light chain. Therapeutic and diagnostic uses of such hybrid antibodies are also disclosed.

### SUMMARY OF THE INVENTION

In general, the invention features, a hybrid antibody molecule, e.g., a hybrid anti-CD3 antibody, which includes a humanized, or CDR-grafted, light chain variable region, and a chimeric heavy chain variable region. Preferably, the hybrid antibody molecule has improved assembly characteristics, e.g., as compared to a fully humanized, or CDR grafted antibody, i.e., an antibody having a CDR-grafted or humanized light, and a CDR-grafted or humanized heavy chain. In one embodiment, the hybrid antibody molecule binds with high affinity and specificity to CD3, preferably human CD3.

In a preferred embodiment, a humanized, or CDR-grafted, light chain variable region includes at least one, preferably two, and more preferably all three complementarity determining regions (CDR's) from a donor immunoglobulin, e.g., a rodent (mouse or rat) immunoglobulin, or from an in vitro generated immunoglobulin, e.g., an immunoglobulin generated by phage display. Preferably, the humanized, or CDR-grafted, light chain variable region framework: (i) is about 85% or more identical, preferably 90%, 95%, 99% or more identical to a corresponding part of an acceptor immunoglobulin framework, e.g., a naturally-occurring immunoglobulin framework (e.g., a human framework), or a consensus framework; (ii) comprises at least about 60, and more preferably about 70 amino acid residues identical to those in the acceptor immunoglobulin light chain variable region framework, e.g., a naturally-occurring immunoglobulin framework (e.g., a human framework) or a consensus framework; or (iii) includes at least 1 and as many as 4, 6, 8, 10 or 20 non-acceptor residues. Such non-acceptor residues can be e.g., donor residues, or generally any residue other than that of the acceptor, e.g., typical residues.

Preferably, the change(s) or replacement(s) in the light chain acceptor framework improves at least one function of the hybrid antibody molecule, such as binding affinity or assembly. Thus, the humanized light chain variable region framework can include one or more replacements of an acceptor amino acid by a different amino acid, e.g., the corresponding donor amino acid, or a more typical amino acid. Preferred framework replacements are located in one or more of the following positions: adjacent (e.g., immediately adjacent or within 2 or 3 residues) to one of the CDR's in the humanized immunoglobulin sequence, or at a residue capable of interacting with one of the CDR's of the humanized immunoglobulin sequence.

In a preferred embodiment, a chimeric heavy chain variable region is chosen from one of the following:

(a) a heavy chain variable region which includes at least one, preferably two, and more preferably all three CDR's, and at least one, two, three, or all four heavy chain variable framework regions (FR's), e.g., FR1, FR2, FR3 and/or FR4, wherein said at least one CDR and said at least one FR have an amino acid sequence at least about 85%, 90%, 95%, 99% or more identical to a corresponding sequence in the donor immunoglobulin;

(b) a heavy chain variable region which includes at least one, preferably two, and more preferably all three CDR's from the donor immunoglobulin and a heavy chain variable framework that differs by at least one, two, three, four, five, ten, but no more than 15 amino acid residues from the donor immunoglobulin;

(c) a heavy chain variable region at least about 85%, 90%, 95%, 99% or more identical to the heavy chain variable region of the donor immunoglobulin;

(d) a heavy chain variable region that differs by at least one, two, three, four, five, ten and no more than 15 amino acid residues from the donor immunoglobulin; or

(e) a heavy chain variable region from the donor immunoglobulin.

In a preferred embodiment, the light and heavy chains of the hybrid antibody molecule associate more strongly and/or produce an antibody with higher binding affinity than the light and heavy chains of a fully CDR-grafted or a humanized antibody, i.e., an antibody having CDR-grafted light and heavy chains, or an antibody having humanized light and heavy chains.

In a preferred embodiment, the donor immunoglobulin is chosen from a rodent, primate, camel, sheep, goat, or a rabbit immunoglobulin, or an immunoglobulin having a sequence at least about 85%, 90%, 95%, 99% or more identical to a naturally-occurring immunoglobulin sequence from the aforesaid species. Preferably, the donor immunoglobulin is a rodent immunoglobulin, e.g., a mouse or a rat immunoglobulin. In other embodiments, the donor immunoglobulin is an in vitro generated immunoglobulin, e.g., an immunoglobulin generated by phage display.

In a preferred embodiment, the acceptor immunoglobulin is a human immunoglobulin, or an immunoglobulin having a sequence at least about 85%, 90%, 95%, 99% or more identical to a naturally-occurring human immunoglobulin sequence. In other embodiments, the acceptor immunoglobulin is a consensus sequence, or a sequence at least about 85%, 90%, 95%, 99% or more identical thereto.

In a preferred embodiment, the heavy chain variable region is from a rodent (e.g., mouse or rat), primate, camel, sheep, goat, or a rabbit immunoglobulin, or an immunoglobulin having a sequence at least about 85%, 90%, 95%, 99% or more identical thereto. In other embodiments, the heavy chain variable region is from an in vitro generated immunoglobulin, e.g., an immunoglobulin generated by phage display, or an immunoglobulin having a sequence at least about 85%, 90%, 95%, 99% or more identical thereto.

In a preferred embodiment, the heavy chain variable region is from a rodent (e.g., mouse or rat), and the acceptor immunoglobulin is human, e.g., a human constant region.

Preferred hybrid antibody molecules bind to a selected antigen with high affinity, e.g., with an affinity constant of at least about 10⁷ M⁻¹, preferably about 10⁸ M⁻¹, and more preferably, about 10⁹ M⁻¹ to 10¹⁰ M⁻¹ or stronger.

The heavy and light chains of the hybrid antibody molecules of the invention can be full-length (e.g., a hybrid antibody molecule can include at least one, and preferably two complete heavy chains, and at least one, and preferably two complete light chains) or can include only an antigen-binding fragment (e.g., a Fab, F(ab′)₂, Fv or a single chain Fv fragment).

The hybrid antibody molecule can include a constant region, or a portion thereof, chosen from any of: the kappa, lambda, alpha, gamma, delta, epsilon and mu constant region genes. For example, heavy chain constant regions of the various isotypes can be used, including: IgG1, IgG2, IgG3, IgG4, IgM, IgA1, IgA2, IgD, and IgE. The light chain constant region can be chosen from kappa or lambda. Preferably, the hybrid antibody molecule is an IgG, more preferably, IgG1. Preferred hybrid antibody molecules have a human type kappa or lambda constant region.

In a preferred embodiment, the heavy or the light chain constant region is from human, primate, camel, sheep, goat, rodent (e.g., rat or mouse), or rabbit origin, or has a sequence at least about 85%, 90%, 95%, 99% or more identical to a naturally-occurring immunoglobulin sequence from the aforesaid species. Preferred heavy or light chain constant region is from human origin, or has a sequence at least about 85%, 90%, 95%, 99% or more identical to a human sequence.

In one exemplary embodiment, the hybrid antibody molecule has a humanized or CDR-grafted light chain, e.g., three rat or mouse CDR's, in a human acceptor framework, and a fully rat or mouse heavy chain variable region, linked to human light and heavy chain constant regions, respectively. In other exemplary embodiments, the hybrid antibody can have humanized or CDR-grafted light chain, e.g., three rat CDR's, in a human acceptor framework and a fully rat heavy chain variable region, linked to mouse light and heavy chain constant regions, respectively.

In a preferred embodiment, a heavy and/or the light chain constant region of the hybrid antibody molecule can be modified by replacing one or more amino acids. In one embodiment, the constant region can be modified to alter (e.g., increase or decrease) one or more of the following: the glycosylation pattern, the Fc receptor binding sites, the ability to fix complement, or the cysteine residues. For example, residues which are part of the N-glycosylation motif, (e.g., asparagine residue at position 297 in the human IgG constant region) can be replaced, e.g., using mutagenesis techniques, with another amino acid that cannot be glycosylated, e.g., alanine. Such modified constant regions have a reduced number of glycosylation sites, and in some embodiments, can be a glycosylated.

The hybrid antibody molecules of the invention can be used as diagnostic or therapeutic agents in vivo and in vitro. A preferred hybrid antibody molecule binds to a leukocyte antigen, e.g., a T cell antigen, or a tumor antigen. For example, the I antigen can be chosen from: CD1, CD2, CD3, CD4, CD5, CD8, CD18, CD20, CD23, CD40L, and CD80. Preferred T cell antigens include CD3, CD18 and CD80. Alternatively, the leukocyte antigen can be a chemokine receptor, e.g., a CXC chemokine receptor or a CC chemokine receptor. Exemplary chemokine receptors include CCR1, CCR2A, CCR2B, CCR3, CCR4, CCR5, CCR6, CCR7, CCR8, CCR9, CCR1O, CXCR1, CXCR2, CXCR3, and CXCR4. Examples of tumor antigen include EGFR, Her2/neu, HELP, GCC, PSMA, PSA, CD66-c, prostasin, TMPRSS3, TADG 12 and TADG 15.

In another aspect, the invention provides, a hybrid anti-CD3 antibody molecule which includes a humanized, or CDR-grafted, light chain variable region, e.g., a humanized or CDR-grafted light chain variable region as described herein, and a chimeric heavy chain variable region, e.g., a chimeric heavy chain variable region as described herein.

In a preferred embodiment, the light and heavy chains of the hybrid anti-CD3 antibody molecule have improved assembly characteristics, e.g., as compared to a fully humanized, or CDR grafted anti-CD3 antibody, i.e., an anti-CD3 antibody having a CDR-grafted or humanized light, and a CDR-grafted or humanized heavy chain, respectively.

In a preferred embodiment, the donor immunoglobulin is a rodent, e.g., a rat or a mouse, immunoglobulin.

In a preferred embodiment, the heavy chain variable region of the anti-CD3 hybrid antibody molecule has at least one, preferably two, and most preferably three, CDR's chosen from the amino acid sequences of SEQ ID NOs:1, 2, and 3, or a sequence which differs by no more than 1 or 2 amino acid residues from SEQ ID NO:1, 2, or 3.

In a preferred embodiment, the light chain variable region of the anti-CD3 hybrid antibody molecule has at least one, preferably two, and most preferably three CDR's chosen from the amino acid sequences of SEQ ID NOs:4, 5, and 6, or a sequence which differs by no more than 1 or 2 amino acid residues from SEQ ID NO:4, 5, or 6.

In a preferred embodiment, the heavy chain and the light chain variable region of the anti-CD3 hybrid antibody molecule has at least one, two, three, four, five and preferably all six CDR's chosen from the amino acid sequences of SEQ ID NOs:1, 2, 3, 4, 5, and 6, or a sequence which differs by no more than 1 or 2 amino acid residues from SEQIDNO:1,2, 3,4,5, or6.

In a preferred embodiment, the heavy chain variable framework region of the anti-CD3 hybrid antibody molecule has at least one, preferably two, three and most preferably four FR's amino acid sequence chosen from SEQ ID NOs:7, 8, 9, and 10, or a sequence which differs by no more than 1, 2, 3 or 4 amino acid residues from SEQ ID NO:7, 8, 9, or 10.

In a preferred embodiment, the light chain variable framework region of the anti-CD3 hybrid antibody molecule has at least one, preferably two, three and most preferably four amino acid sequence chosen from SEQ ID NO:11, 12, 13, or 14, or a sequence which differs by no more than 1, 2, 3 or 4 amino acid residues from SEQ ID NO:11, 12, 13, or 14.

In a preferred embodiment, the heavy chain variable region of the anti-CD3 hybrid antibody molecule has the amino acid sequence shown in SEQ ID NO:17, or a sequence at least about 85%, 90%, 95%, 99% or more identical thereto, or which differs by no more than 1, 2, 5, 10, or 15 amino acid residues from SEQ ID NO:17.

In a preferred embodiment, the light chain variable region of the anti-CD3 hybrid antibody molecule has the amino acid sequence shown in SEQ ID NO:15, or a sequence at least about 85%, 90%, 95%, 99% or more identical thereto, or which differs by no more than 1, 2, 5, 10, or 15 amino acid residues from SEQ ID NO:15.

In a preferred embodiment, the light chain variable region of the anti-CD3 hybrid antibody molecule is linked to a human type lambda constant region.

In a preferred embodiment, the heavy chain variable region of the anti-CD3 hybrid antibody molecule is linked to a heavy chain constant region of an IgG, e.g., an IgG1, isotype.

In a preferred embodiment, the light chain variable region of the anti-CD3 hybrid antibody molecule is linked to a human type lambda constant region, and the heavy chain variable region of the anti-CD3 hybrid antibody molecule is linked to a heavy chain constant region of an IgG, e.g., an IgG1, isotype

In a preferred embodiment, the heavy or the light chain constant region of the anti-CD3 antibody molecule is modified by replacing one or more amino acids. For example, the constant region can be modified by altering one or more of the following: the glycosylation sites, the Fc receptor binding sites, the complement fixation sites, or the cysteine residues. For example, the asparagine residue at position 297 of the constant region can be modified to render an aglycosylated antibody.

In another aspect, the invention provides, compositions, e.g., pharmaceutical compositions, which include a pharmaceutically acceptable carrier and at least one of the hybrid antibody molecules described herein (e.g., a hybrid anti-CD3 antibody molecule described herein). In one embodiment, the compositions, e.g., pharmaceutical compositions, comprise a combination of two or more one of the aforesaid hybrid antibody molecules. For example, a composition, e.g., pharmaceutical composition, comprising an anti-CD3 antibody molecule, in combination with another T cell- or tumor cell-specific antigen. Combinations of the hybrid antibody molecule and a drug, e.g., a therapeutic agent (e.g., a cytototoxic or cytostatic drug), are also within the scope of the invention.

The hybrid antibody molecules (e.g., the hybrid anti-CD3 antibody molecule) described herein can be derivatized or linked to another functional molecule, e.g., another peptide or protein (e.g., an Fab′ fragment). For example, an antibody, or antigen-binding portion, of the invention can be functionally linked (e.g., by chemical coupling, genetic fusion, non-covalent association or otherwise) to one or more other molecular entities, such as another antibody (e.g., a bispecific or a multispecific antibody), toxins, radioisotopes, cytotoxic or cytostatic agents, among others.

The invention also features nucleic acid sequences which encode a heavy and light chain described herein. For example, the invention features, a first and second nucleic acid encoding heavy and light chain variable region, respectively, of a hybrid antibody molecule (e.g., a hybrid anti-CD3 antibody), wherein the heavy chain variable region is a chimeric heavy chain variable region, e.g., a chimeric heavy chain variable region as described herein, and the light chain variable region is a humanized or CDR-grafted light chain variable region, e.g., a humanized or CDR-grafted light chain variable region as described herein.

In another aspect, the invention features host cells and vectors containing the nucleic acids of the invention.

In another aspect, the invention features, a method of providing a hybrid antibody molecule, e.g., a hybrid antibody molecule having improved assembly characteristics, comprising:

providing a first nucleic acid encoding a chimeric heavy chain variable region, e.g., a chimeric heavy chain variable region as described herein;

providing a second nucleic acid encoding a humanized or CDR-grafted light chain variable region, e.g., a humanized or CDR-grafted light chain variable region as described herein; and

introducing said first and second nucleic acids into a host cell under conditions that allow expression and assembly of said light and heavy chain variable regions.

The first and second nucleic acids can be linked or unlinked, e.g., expressed on the same or different vector, respectively.

In a preferred embodiment, the host cell is a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. For example, nucleic acids encoding the hybrid antibody molecules described herein can be expressed in a transgenic animal. In one embodiment, the nucleic acids are placed under the control of a tissue-specific promoter (e.g., a mammary specific promoter) and the antibody is produced in the transgenic animal. For example, the hybrid antibody molecule is secreted into the milk of the transgenic animal, such as a transgenic cow, pig, horse, sheep, goat or rodent.

Yet another aspect of the invention pertains to methods for modulating the activity of an aberrant (e.g., hyperproliferative) cell, e.g., an aberrant (e.g., hyperproliferative) immune or a cancer cell, or modulating the expression or function of a selected antigen. The method includes contacting the aberrant cell, or the selected antigen, with a hybrid antibody molecules described herein, such that the activity of the aberrant cell, or the expression or function of the antigen, is modulated. The subject method can be used on cells in culture, e.g. in vitro or ex vivo. For example, immune or cancer cells can be cultured in vitro in culture medium and the contacting step can be effected by adding the hybrid antibody molecule, to the culture medium. The method can be performed on cells (e.g., immune or cancer cells) present in a subject, e.g., as part of an in vivo (e.g., therapeutic or prophylactic) protocol.

For in vivo methods, the hybrid antibody molecule, alone or in combination with another agent, can be administered to a subject, e.g., a human, suffering from a disorder, e.g., an immune disorder (e.g., a T cell mediated-disorder) or a cancer, in an amount sufficient to ameliorate or prevent said disorder.

Exemplary immune disorders that can be treated (e.g., ameliorated) or prevented using the methods and compositions of the invention include, for example, transplant rejection or autoimmune disorders (e.g., including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis)).

Exemplary cancer disorders include, but are not limited to, a solid tumor, a soft tissue tumor (e.g., a lymphoma or a leukemia), and a metastatic lesion.

In yet another aspect, the invention provides a method for detecting the presence of a selected antigen, e.g., an antigen recognized by a hybrid antibody molecule, in a sample, in vitro (e.g., a biological sample, such as serum, plasma, tissue, biopsy). The subject method can be used to diagnose a disorder, e.g., an immune (e.g., a T cell disorder) or a cancer. The method includes: (i) contacting the sample or a control sample with the hybrid antibody molecule; and (ii) detecting formation of a complex between the hybrid antibody molecule, and the sample or the control sample, wherein a statistically significant change in the formation of the complex in the sample relative to the control sample is indicative of the presence of the antigen in the sample.

In yet another aspect, the invention provides a method for detecting the presence of an antigen recognized by a hybrid antibody molecule, in vivo (e.g., in vivo imaging in a subject). The subject method can be used to diagnose a disorder, e.g., an immune (e.g., a T cell) disorder or a cancer. The method includes: (i) administering the hybrid antibody molecule to a subject or a control subject under conditions that allow binding of the hybrid antibody molecule to a selected antigen; and (ii) detecting formation of a complex between the hybrid antibody molecule and the selected antigen, wherein a statistically significant change in the formation of the complex in the subject relative to the control subject is indicative of the presence of the antigen.

Preferably, the hybrid antibody molecule is directly or indirectly labeled with a detectable substance to facilitate detection of the bound or unbound antibody. Suitable detectable substances include various enzymes, prosthetic groups, fluorescent materials, luminescent materials and radioactive materials.

Other features and advantages of the instant invention will become more apparent from the following detailed description and claims.

### Brief Description of the Drawings

FIG. 1 depicts the nucleotide and amino acid sequences of the light chain variable region of the humanized anti-human CD3 antibody (SEQ ID NOs:16 and 15, respectively).

FIG. 2 depicts the nucleotide and amino acid sequences of the heavy chain variable region of the rat anti-human CD3 antibody (SEQ ID NOs: 18 and 17, respectively).

FIGS. 3A-3B depict the nucleotide and amino acid sequence of the human IgGI (SEQ ID NOs:26 and 25, respectively) constant region with a mutation at amino acids 297-299 to remove the glycosylation site.

### Detailed Description of the Invention

This invention pertains to hybrid antibody molecules, which include a humanized or CDR-grafted light chain variable region, and a chimeric heavy chain variable region. Preferably, the light and heavy immunoglobulin chains of the hybrid antibodies of the present invention have improved assembly characteristics, compared, e.g., to a fully humanized, or CDR grafted antibody, i.e., an antibody having a CDR-grafted or humanized light, and a CDR-grafted or humanized heavy chain, respectively. In one embodiment, the hybrid antibody binds with high affinity to CD3. Accordingly, various aspects of the invention relate to hybrid antibody molecules, pharmaceutical compositions thereof, nucleic acids encoding the aforesaid antibody molecules, as well as vectors and host cells containing the aforesaid nucleic acid sequences. Methods of producing the aforesaid hybrid antibody molecules, as well as methods of using the antibodies of the invention to detect a selected antigen, e.g., CD3, or to modulate the activity expressing the selected antigen, either in vitro or in vivo, are also encompassed by the invention.

In order that the present invention may be more readily understood, certain terms are first defined. Additional definitions are set forth throughout the detailed description.

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

As used herein, the term “immunoglobulin” refers to a protein consisting of one or more polypeptides substantially encoded by immunoglobulin genes. The recognized human immunoglobulin genes include the kappa, lambda, alpha (Iga1 and IgA2), gamma (IgGa1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH--terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

As used herein, “isotype” refers to the antibody class (e.g., IgM or IgG1) that is encoded by heavy chain constant region genes.

The term “antigen-binding fragment” of an antibody (or simply “antibody portion,” or “fragment”), as used herein, refers to one or more fragments of a full-length antibody that retain the ability to specifically bind to an antigen (e.g., CD3). Examples of binding fragments encompassed within the term “antigen-binding fragment” of an antibody include (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

As used herein, a “chimeric immunoglobulin heavy chain” refers to those immunoglobulin heavy chains having a portion of the immunoglobulin heavy chain, e.g., the variable region, at least 85%, preferably, 90%, 95%, 99% or more identical to a corresponding amino acid sequence in an immunoglobulin heavy chain from a particular species, or belonging to a particular antibody class or type, while the remaining segment of the immunoglobulin heavy chain (e.g., the constant region) being substantially identical to the corresponding amino acid sequence in another immunoglobulin molecule. For example, the heavy chain variable region has a sequence substantially identical to the heavy chain variable region of an immunoglobulin from one species (e.g., a “donor” immunoglobulin, e.g., a rodent immunoglobulin), while the constant region is substantially identical to the constant region of another species immunoglobulin (e.g., an “acceptor” immunoglobulin, e.g., a human immunoglobulin). In one embodiment, the donor immunoglobulin is an in vitro generated immunoglobulin, e.g., an immunoglobulin generated by phage display.

As used herein, the term “humanized” or “CDR-grafted” light chain variable region refers to an immunoglobulin light chain comprising one or more CDR's, or having an amino acid sequence which differs by no more than 1 or 2 amino acid residues to a corresponding one or more CDR's from one species, or antibody class or type, e.g., a “donor” immunoglobulin (e.g., a non-human (usually a mouse or rat) immunoglobulin, or an in vitro generated immunoglobulin); and a framework region having an amino acid sequence about 85% or higher, preferably 90%, 95%, 99% or higher identical to a corresponding part of an acceptor immunoglobulin framework from a different species, or antibody class or type, e.g., a naturally-occurring immunoglobulin framework (e.g., a human framework) or a consensus framework. In some embodiments, the framework region includes at least about 60, and more preferably about 70 amino acid residues identical to those in the acceptor immunoglobulin light chain variable region framework, e.g., a naturally-occurring immunoglobulin framework (e.g., a human framework) or a consensus framework.

Typically, the immunoglobulin providing the CDR's is called the “donor” and the immunoglobulin providing the framework is called the “acceptor.” In one embodiment, the donor immunoglobulin is a non-human (e.g., rodent), or an in vitro generated immunoglobulin, e.g., an immunoglobulin generated by phage display. The acceptor framework is a naturally-occurring (e.g., a human) framework or a consensus framework, or a sequence about 85% or higher, preferably 90%, 95%, 99% or higher identical thereto.

The light chain variable region may have replacements in only one or more of the CDR's, and thus will be referred to herein as a “CDR-grafted” light variable chain. In other embodiments, it may include framework substitutions, in addition to the CDR substitutions, which will be referred to herein as a “humanized” light chain variable region.

Constant regions need not be present, but if they are, they can be identical or substantially identical to the acceptor (e.g., human immunoglobulin) constant regions, or identical or substantially identical to a constant region from a third species of antibody class. In one exemplary embodiment, the light chain CDR's are from a rat immunoglobulin light chain, the framework regions are from a human immunoglobulin light chain, and the constant region is from a mouse immunoglobulin.

A “hybrid antibody molecule” refers to an antibody, or an antigen-binding fragment thereof (e.g., a Fab, F(ab′)₂, Fv or a single chain Fv fragment), which includes a humanized, or CDR-grafted, light chain variable region, and a chimeric heavy chain variable region. Each heavy and light chain variable region of the hybrid antibody may, optionally, include a corresponding constant, which can be identical or similar (e.g., about 85% or higher, preferably 90%, 95%, 99% or higher) to the acceptor constant regions (e.g., human immunoglobulin, or a constant region from yet another species, or antibody class or type). The term “hybrid antibody” or “hybrid antibody molecule” does not encompass a typical chimeric antibody, e.g., an antibody whose light and heavy chains are obtained from immunoglobulin variable and constant region genes belonging to different species or class, or a typical humanized antibody, e.g., an antibody whose light and heavy chain CDR's belong to different species or class from the framework regions.

As used herein, “an in vitro generated” “antibody” or “immunoglobulin” refers to an immunoglobulin where all or part of the variable region, e.g., one or more or all CDR's, is generated in a non-immune cell selection, e.g., an in vitro phage display, protein chip or any other method in which candidate sequences can be tested for their ability to bind to an antigen. This term excludes sequences generated by genomic rearrangement in an immune cell. In one embodiment, a donor immunoglobulin sequence of a hybrid antibody molecule described herein can be in vitro generated. The invention also pertains to hybrid antibody molecules which include a chimeric in vitro generated light chain variable region and a humanized heavy chain variable region, wherein the donor is an in vitro generated antibody.

As used herein, the term “substantially identical,” or “sufficiently identical,” (or “substantially” or “sufficiently” “homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the same.

Sequences similar or homologous (e.g., at least about 85% sequence identity) to the sequences disclosed herein are also part of this application. In some embodiment, the sequence identity can be about 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453 ) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http:/Hwww.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6×sodium chloride/sodium citrate (SSC) at about 45° C., followed by one or more washes in 0.2×SSC, 0.1% SDS at 50° C. Another example of stringent hybridization conditions are hybridization in 6×SSC at about 45° C., followed by one or more washes in 0.2×SSC, 0.1% SDS at 55° C. A further example of stringent hybridization conditions are hybridization in 6×SSC at about 45° C., followed by one or more washes in 0.2×SSC, 0.1% SDS at 60° C. Preferably, stringent hybridization conditions are hybridization in 6×SSC at about 45° C., followed by one or more washes in 0.2×SSC, 0.1% SDS at 65° C. Particularly preferred highly stringent conditions (and the conditions that should be used if the practitioner is uncertain about what conditions should be applied to determine if a molecule is within a hybridization limitation of the invention) are 0.5M sodium phosphate, 7% SDS at 65° C., followed by one or more washes at 0.2×SSC, 1% SDS at 65° C.

It is understood that the hybrid antibodies of the present invention may have additional conservative or non-essential amino acid substitutions, which do not have a substantial effect on antigen binding or other immunoglobulin functions.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of a hybrid antibody, without abolishing or more preferably, without substantially altering a biological activity, whereas an “essential” amino acid residue results in such a change.

The hybrid antibody molecules may have an immunoglobulin sequence that differs by, e.g., at least one, two, three, four, five, ten and no more than a given number of amino acid residues from another sequence. As used herein, the term “differs” includes differences amino acid sequences created by, e.g., deletions, insertions, or substitutions, of residues of the known amino acid sequence of a protein. “Looped” out sequences from deletions or insertions, or mismatches, are considered differences. For example, residues are counted as differences when the humanized immunoglobulin sequence results from a replacement of an amino acid residue in the acceptor immunoglobulin by another residue, e.g., a replacement of an amino acid in the acceptor for the corresponding donor residue or a more typical residue. No differences are counted when the acceptor and donor sequences have the same residue at the corresponding position.

The term “from” when used to refer to a region or sequence (e.g., a CDR or framework region) from a donor refers to synthetic, as well as recombinantly-produced sequences. The term “from” refers to biological origin or sequence relatedness.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

As used herein, a more “typical” amino acid residue in an immunoglobulin refers to a residue that occurs in more than about 50% of the sequences in a representative databank. An “unusual” or “rare” amino acid residue occurs less than about 20%, typically less than 10% of the sequences. When deciding whether an amino acid in an acceptor, e.g., a human acceptor, is “rare” or “typical” among acceptor, e.g., human sequences, it is preferable to consider only those sequences present in the same subgroup as the acceptor sequence (see Kabat et al. supra).

An “isolated” or “purified” polypeptide or protein, e.g., an “isolated antibody,” is substantially free of cellular material or other contaminating proteins from the cell or tissue source from which the protein is derived, or substantially free from chemical precursors or other chemicals when chemically synthesized. In preferred embodiments, the preparation of antibody protein having less than about 30% is considered to be “substantially free.” In a preferred embodiment, 20%, 10% and more preferably 5% (by dry weight), of non-antibody protein (also referred to herein as a “contaminating protein”), or of chemical precursors. When the antibody protein or biologically active portion thereof is recombinantly produced, it is also preferably substantially free of culture medium, i.e., culture medium represents less than about 20%, more preferably less than about 10%, and most preferably less than about 5% of the volume or mass of the protein preparation. The invention includes isolated or purified preparations of at least 0.01 milligrams in dry weight. In a preferred embodiment, the preparations are 0.1, 1.0, and 10 milligrams in dry weight.

The term “isolated antibody”, as used herein, is also intended to refer to an antibody that is substantially free of other antibodies having different antigenic specificities (e.g., an isolated antibody that specifically binds to a CD3 antigen is substantially free of antibodies that specifically binds antigens other than CD3; or an isolated bispecific antibody that binds to CD3 and CD8 is substantially free of antibodies that specifically binds antigens other than CD3 or CD8).

As used herein, the term “assembly characteristics” refers to one or more of the following properties: (1) dimer or tetramer formation; (2) percentage of properly folded antibody, e.g., formation of correct disulfide bonds; (3) binding affinity and/or specificity; (4) yield of functional antibody, as measured by, e.g., binding affinity; or (5) high levels of antibody production, e.g., at least from about 10 μg/ml, preferably, 100 μ/ml, more preferably 800 μ/ml, and yet more preferably 1.5 mg/ml or higher production levels. A hybrid antibody molecule shows improved antibody characteristics when one or more of the aforesaid antibody properties is increased, e.g., shows a statistically significant increase, relative to a fully chimeric or humanized antibody made under the same conditions.

As used herein, “specific binding” refers to the property of the antibody: (1) to bind to a predetermined antigen with an affinity of at least 1×10⁷ M⁻¹, and (2) to preferentially bind to the predetermined antigen with an affinity that is at least two-fold greater than its affinity for binding to a non-specific antigen (e.g., BSA, casein) other than the predetermined antigen. The phrases “an antibody recognizing an antigen” and “an antibody specific for an antigen” are used interchangeably herein with the term “an antibody which binds specifically to an antigen”.

The term “K_{assoc}”. as used herein, is intended to refer to the association constant of a particular antibody-antigen interaction.

The term “K_{dis}”, as used herein, is intended to refer to the dissociation constant of a particular antibody-antigen interaction.

The term “glycosylation pattern” is defined as the pattern of carbohydrate units that are covalently attached to a protein, more specifically to an immunoglobulin protein. In some embodiments, the glycosylation pattern of a hybrid antibody can be characterized as being substantially similar to glycosylation patterns of naturally occurring antibodies. In other embodiments, the glycosylation pattern may be altered (e.g., reduced or increased) by recombinant or chemical methods. For example, residues which are part of the N-glycosylation motif, Asn-X-Ser, wherein X can be any amino acid residue except proline (e.g., asparagine residue at position 297 in the human IgG constant region) can be replaced, e.g., using mutagenesis techniques, with another amino acid that cannot be glycosylated, e.g., alanine. Such modified constant regions have a reduced number of glycosylation sites, and in some embodiments, can be aglycosylated.

The term “naturally-occurring” as used herein as applied to an object refers to the fact that an object can be found in nature. For example, a polypeptide (e.g., an antibody) or polynucleotide sequence that is present in an organism (including viruses) that can be isolated from a source in nature and which has not been intentionally modified by man in the laboratory is naturally-occurring.

The term “isolated nucleic acid”, as used herein in reference to nucleic acids encoding hybrid antibodies or antibody fragments (e.g., VH, VL, CDR3), is intended to refer to a nucleic acid molecule in which the nucleotide sequences encoding the antibody or antibody portion are free of other nucleotide sequences encoding other antibodies or antibody portions, which other sequences may naturally flank the nucleic acid in human genomic DNA. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The term “recombinant host cell” (or simply “host cell”), as used herein, is intended to refer to a cell into which a recombinant expression vector has been introduced. It should be understood that such terms are intended to refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein.

Various aspects of the invention are described in further detail in the following subsections.

Production of Hybrid Antibody Molecules

The hybrid antibody molecules can be generated using art-recognized techniques for producing chimeric and humanized immunoglobulin chains, as described in detail below. The antibodies can be of the various isotypes, including: IgG (e.g., IgG1, IgG2, IgG3, IgG4), IgM, IgA1, IgA2, IgD, or IgE. Preferably, the antibody is an IgG isotype. The antibody molecules can be full-length (e.g., an IgG1 or IgG4 antibody) or can include only an antigen-binding fragment (e.g., a Fab, F(ab′)₂, Fv or a single chain Fv fragment).

As described in more detail below, antibodies (preferably, monoclonal antibodies from differing organisms, e.g., rodent, sheep, human) against a predetermined antigen can be produced using art-recognized methods. Once the antibodies are obtained, the variable regions can be sequenced. The location of the CDR's and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). The light and heavy chain variable regions can, optionally, be ligated to corresponding constant regions. A chimeric and a humanized immunoglobulin chain can be generated and co-expressed into the appropriate host cells.

Monoclonal antibodies (e.g., in vitro-generated recombinant antibodies) can be produced by a variety of techniques, including conventional monoclonal antibody methodology e.g., the standard somatic cell hybridization technique of Kohler and Milstein, Nature 256: 495 (1975). Although somatic cell hybridization procedures are preferred, in principle, other techniques for producing monoclonal antibody can be employed e.g., viral or oncogenic transformation of B lymphocytes. The preferred animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

Human monoclonal antibodies (mAbs) directed against human proteins can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al.,1991, Biotechniques 11: 152-156). A similar strategy can also been used to amplify human heavy and light chain variable regions from human antibodies (Larrick et al., 1991, Methods: Companion to Methods in Enzymology 2:106-110).

In an illustrative embodiment, RNA is isolated from B lymphocytes, for example, peripheral blood cells, bone marrow, or spleen preparations, using standard protocols (e.g., U.S. Pat. No. 4,683,202; Orlandi, et al. PNAS (1989) 86:3833-3837; Sastry et al., PNAS (1989) 86:5728-5732; and Huse et al. (1989) Science 246:1275-1281.) First-strand cDNA is synthesized using primers specific for the constant region of the heavy chain(s) and either of the κ and λ light chains, as well as primers for the signal sequence. Using variable region PCR primers, the variable regions of both heavy and light chains are amplified, each alone or in combination, and ligated into appropriate vectors for further manipulation in generating the display packages. Oligonucleotide primers useful in amplification protocols may be unique or degenerate or incorporate inosine at degenerate positions. Restriction endonuclease recognition sequences may also be incorporated into the primers to allow for the cloning of the amplified fragment into a vector in a predetermined reading frame for expression.

The V-gene library cloned from the immunization-derived antibody repertoire can be expressed by a population of display packages, preferably derived from filamentous phage, to form an antibody display library. Ideally, the display package comprises a system that allows the sampling of very large variegated antibody display libraries, rapid sorting after each affinity separation round, and easy isolation of the antibody gene from purified display packages. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating a variegated antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

Once displayed on the surface of a display package (e.g., filamentous phage), the antibody library is screened with the antigen, or peptide fragment thereof, to identify and isolate packages that express an antibody having specificity for the antigen. Nucleic acid encoding the selected antibody can be recovered from the display package (e.g., from the phage genome) and subcloned into other expression vectors by standard recombinant DNA techniques.

Specific antibodies with high affinities for a surface protein can be made according to methods known to those in the art, e.g, methods involving screening of libraries (Ladner, R. C., et al., U.S. Pat. No. 5,233,409; Ladner, R. C., et al., U.S. Pat. No. 5,403,484). Further, the methods of these libraries can be used in screens to obtain binding determinants that are mimetics of the structural determinants of antibodies.

In particular, the Fv binding surface of a particular antibody molecule interacts with its target ligand according to principles of protein-protein interactions, hence sequence data for V_{H} and V_{L} (the latter of which may be of the κ or λ chain type) is the basis for protein engineering techniques known to those with skill in the art. Details of the protein surface that comprises the binding determinants can be obtained from antibody sequence information, by a modeling procedure using previously determined three-dimensional structures from other antibodies obtained from NMR studies or crytallographic data. See for example Bajorath, J. and S. Sheriff, 1996, Proteins: Struct., Funct., and Genet. 24 (2), 152-157; Webster, D. M. and A. R. Rees, 1995, “Molecular modeling of antibody-combining sites,” in S. Paul, Ed., Methods in Molecular Biol. 51, Antibody Engineering Protocols, Humana Press, Totowa, N.J., pp 17-49; and Johnson, G., Wu, T. T. and E. A. Kabat, 1995, “Seqhunt: A program to screen aligned nucleotide and amino acid sequences,” in Methods in Molecular Biol.51, op. cit., pp 1-15.

An antigen binding region can also be obtained by screening various types of combinatorial libraries with a desired binding activity, and to identify the active species, by methods that have been described.

In one embodiment, a variegated peptide library is expressed by a population of display packages to form a peptide display library. Ideally, the display package comprises a system that allows the sampling of very large variegated peptide display libraries, rapid sorting after each affinity separation round, and easy isolation of the peptide-encoding gene from purified display packages. Peptide display libraries can be in, e.g., prokaryotic organisms and viruses, which can be amplified quickly, are relatively easy to manipulate, and which allows the creation of large number of clones. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages. Phage display libraries are described above.

Other techniques include affinity chromatography with an appropriate “receptor” to isolate binding agents, followed by identification of the isolated binding agents or ligands by conventional techniques (e.g., mass spectrometry and NMR). Preferably, the soluble receptor is conjugated to a label (e.g., fluorophores, calorimetric enzymes, radioisotopes, or luminescent compounds) that can be detected to indicate ligand binding. Alternatively, immobilized compounds can be selectively released and allowed to diffuse through a membrane to interact with a receptor.

Combinatorial libraries of compounds can also be synthesized with “tags” to encode the identity of each member of the library (see, e.g., W.C. Still et al., International Application WO 94/08051). In general, this method features the use of inert but readily detectable tags, that are attached to the solid support or to the compounds. When an active compound is detected, the identity of the compound is determined by identification of the unique accompanying tag. This tagging method permits the synthesis of large libraries of compounds which can be identified at very low levels among to total set of all compounds in the library.

Chimeric antibodies, including chimeric immunoglobulin chains, can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

An antibody or an immunoglobulin chain can be humanized by methods known in the art. Humanized antibodies, including humanized immunoglobulin chains, can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target. The recombinant DNA encoding the humanized antibody, or fragment thereof, can then be cloned into an appropriate expression vector.

Humanized or CDR-grafted antibody molecules or immunoglobulins can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference. All of the CDR's of a particular human antibody may be replaced with at least a portion of a non-human CDR or only some of the CDR's may be replaced with non-human CDR's. It is only necessary to replace the number of CDR's required for binding of the humanized antibody to a predetermined antigen.

Also within the scope of the invention are humanized antibodies, including immunoglobulins, in which specific amino acids have been substituted, deleted or added. In particular, preferred humanized antibodies have amino acid substitutions in the framework region, such as to improve binding to the antigen. For example, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the contents of which are hereby incorporated by reference. Other techniques for humanizing immunoglobulin chains, including antibodies, are described in Padlan et al. EP 519596 A1, published on Dec. 23, 1992.

In one embodiment, the present invention provides hybrid anti-CD3 antibodies. Methods for producing fully humanized anti-CD3 antibodies are described in Bolt, S. et al. (1993) Eur. J. Immunol. 23(2):403-1 1; Routledge, E.G. et al. (1995) Transplantation 60(8):847-53; U.S. Pat. Nos. 5,585,097; 5,968,509, the contents of all of which are hereby incorporated by reference. Three humanized light chains are described briefly below and in further detail in the appended examples. For example, an M7 mutant light chain, which includes a humanized light chain variable region (V_{L}), can be generated by replacing all three human CDR's of the human HUMIGHAT LV6C with three CDR's from the light chain of YTH 12.5, a rat anti-CD3 antibody. Mutant 7 contains at least two additional changes, at residues 2 and 4 (according to Kabat numbering), in the light chain acceptor framework. Residue 2 in the acceptor framework can be replaced with the corresponding donor residue (Phe to Ala). Residue 4 in the acceptor framework can be replaced with the corresponding donor residue (Leu to Val). Another light chain mutant, the M8 mutant light chain includes a humanized light chain variable region (V_{L}) which can be generated by replacing all three human CDR's of the human HUMIGHAT LV6C with three CDR's from the light chain of YTH 12.5. Mutant 8 includes one additional change in the light chain acceptor framework. Residue 46 in the acceptor can be replaced with a non-donor residue (Thr to Leu). A third mutant, the M9 mutant light chain can be generated by replacing all three human CDR's of the human HUMIGHAT LV6C with three CDR's from the light chain of YTH 12.5, a rat anti-CD3 antibody. Mutant 9 contains three additional acceptor framework changes. The donor residue is used at position 2 of the acceptor framework (Phe to Ala) and at position 4 of the acceptor framework (Leu to Val). A non-donor residue is used at residue 46 of the acceptor framework (Thr to Leu). In other words, M9 includes all changes present in M7 and M8.

Monoclonal, chimeric and humanized antibodies, which have been modified by, e.g., deleting, adding, or substituting other portions of the antibody, e.g., the constant region, are also within the scope of the invention. For example, an antibody can be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others.

In one embodiment, the constant region of the hybrid antibody can be replaced by another constant region from, e.g., a different species. This replacement can be carried out using molecular biology techniques. For example, the nucleic acid encoding the VL or VH region of a hybrid antibody can be converted to a full-length light or heavy chain gene, respectively, by operatively linking the VH or VL-encoding nucleic acid to another nucleic acid encoding the light or heavy chain constant regions. The sequences of human light and heavy chain constant region genes are known in the art. Preferably, the constant region is human, but constant species from other species, e.g., rodent (e.g., mouse or rat), primate, camel, rabbit can also be used. Constant regions from these species are known in the art (see e.g., Kabat, E. A., et al. (1991) Sequences of Proteins of Immunological Interest, Fifth Edition, U.S. Department of Health and Human Services, NIH Publication No. 91-3242).

Modified hybrid antibody molecules may have enhanced therapeutic applications compared to their unmodified counterparts. For example, as described below, aglycosylated CD3 antibodies which have a modified Fc region, have been shown to be substantially non-mitogenic to T cells, while retaining immunosuppressive properties.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and 5,648,260, the contents of all of which are hereby incorporated by reference). Similar type of alterations could be described which if applied to the murine, or other species immunoglobulin would reduce or eliminate these functions.

For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

In other embodiments, replacing residue 297 (asparagine) with alanine in the IgG constant region significantly inhibits recruitment of effector cells, while only slightly reducing (about three fold weaker) affinity for Clq (see e.g., U.S. Pat. No. 5,624,821). The numbering of the residues in the immunoglobulin chain is that of the EU index (see Kabat et al., 1991). This alteration destroys the glycosylation site and it is believed that the presence of carbohydrate is required for Fc receptor binding. The modification at residue 297 (asparagine to alanine) has been shown to produce aglycosylated anti-CD3 antibodies of the IgG subclass having significantly reduced binding of the antibody Fc region to the Fc receptor. Aglycosylated CD3 antibodies have been shown to be substantially non-mitogenic for human T cells, while being retaining immunosuppressive properties (Bolt, S. et al. (1993) Eur. J. Immunol. 23(2):403-11; Routledge, E. G. et al. (1995) Transplantation 60(8):847-53; U.S. Pat. No. 5,585,097; U.S. Pat5,968,509, the contents of all of which are hereby incorporated by reference). When used as human therapeutics, such aglycosylated antibodies show reduced “first dose effect,” which is a syndrome experienced by patients following the initial administration of the CD3 antibody. This phenomenon requires the cross-linking of the CD3 antigen on the surface of T-cells to accessory cells through Fc receptors. Aglycosylated anti-CD3 antibodies (and in particular, humanized anti-CD3 antibodies) have been shown to elicit a reduced first dose effect, and thus have been shown to be useful therapeutic agents to treat a variety of immune conditions.

Any other substitution at this site that destroys the glycosylation site are believed cause a similar decrease in lytic activity. Other amino acids substitutions, e.g., changing any one of residues 318 (Glu), 320 (Lys) and 322 (Lys), to Ala, are also known to abolish Clq binding to the Fc region of IgG antibodies (see e.g., U.S. Pat. No. 5,624,821).

Modified antibodies can be produced which have a reduced interaction with an Fc receptor. For example, it has been shown that in human IgG3, which binds to the human Fc gamma R1 receptor, changing Leu 235 to Glu destroys the interaction, of the mutant for the receptor. Mutations on adjacent or close sites in the hinge link region of an antibody (e.g., replacing residues 234, 236 or 237 by Ala) can also be used to affect the affinity for the Fc gamma R1 receptor. The numbering of the residues in the immunoglobulin chain is that of the EU index (see Kabat et al., 1991).

Additional methods for altering the lytic activity of an antibody, for example, by altering one or more amino acids in the N-terminal region of the CH2 domain are described in WO 94/29351 by Morgan et al. and U.S. Pat. No. 5,624,821, the contents of all of which are hereby expressly incorporated by reference.

Hybrid antibody fragments comprising only a portion of the primary antibody structure can also be produced, which fragments possess one or more immunoglobulin activities (e.g., antigen binding, complement fixation activity). These polypeptide fragments may be produced by proteolytic cleavage of intact antibodies by methods known in the art, or by inserting stop codons at the desired locations in the vectors using site-directed mutagenesis, such as after CH1 to produce Fab fragments or after the hinge region to produce (Fab′)2 fragments. Single chain antibodies may be produced by joining VL and VH with a DNA linker (see, Huston et al., op. cit., and Bird et al., op. cit.).

A hybrid antibody molecule can be derivatized or linked to another functional molecule (e.g., another peptide or protein). Accordingly, the antibodies and antibody portions of the invention are intended to include derivatized and otherwise modified forms of the hybrid antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized (or labeled) to include fluorescent compounds, various enzymes, prosthetic groups, luminescent materials, bioluminescent materials, and radioactive materials. Labeled antibodies can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with a prostetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The methods of the producing the hybrid antibodies molecules of the present invention can be used produce a variety of antibodies, especially antibodies reactive with markers on cells responsible for a disease. In one embodiment, the hybrid antibodies bind to cancer antigens or immune cell antigens.

Non-limiting examples of cancer antigens recognized by the hybrid antibody molecules of the invention include, but are not limited to, cancer cell antigens from breast, ovary, testis, lung, colon, rectum, pancreas, liver, central nervous system, head and neck, kidney, bone, and soft-tissue tumors (e.g., blood and lymphatic system cancers). Exemplary cancer cell antigens include HELP, GCC, PSMA (prostate specific membrane antigen), PSA (prostate specific antigen), members of the human EGF-like receptor family (e.g., an EGF receptor), HER-2/neu, HER-3, HER-4, carcinoembryonic antigen, gastrin releasing peptide receptor antigen, TAG 72, CD66-c, prostasin, TMPRSS3, TADG 12 (described in U.S. Patent No. 5,972,616) and TADG 15 (described in U.S. Ser. No. 09/261,416).

Non-limiting examples of immune cell antigens include, but are not limited to, antigens present on the surface of erythroid, myeloid, lymphoid lineages, or precursor cells thereof. For example, suitable antigens which bind to immune cells include, but are not limited to, CD1 (a, b, c, d, e), CD2, CD3 (epsilon (ε), gamma (γ), delta (δ)) preferably, CD3 epsilon, CD4, CD5, CD8 (alpha, beta), CD9, CD 10, CD1 1 (a, b, c), CD13, CD14, CD15, CD16, CD18, CD21, CD24, CD29, CD30, CD31, CD34, CD36, CD39, CD40, CD40L, CD41, CD43, CD44, CD44R, CD45, CD47, CD49 (a, b, c, d, e, f), CD50, CD51, CDw52, CD54, CD56, CD57, CD58, CD61, CD62 (e, 1), CD63, CD66, CD70, CD77, CD80, CD86, CD101, CD102, CD103, CD104, CD105, CD106, CD107(a, b), CD121a, CDwl2lb, CD122, CD123, CD124, CDw125, CD126, CD127, CDw128 (a, b), CD152, CD153, CD154, CD158 (a, b), CD166 (see, Leukocyte Typing. (1981). Bernard, A. et al., Eds., Springer-Verlag; Leukocyte Typing II (Vol. I, II, and III). Human Leukocyte Differentiation Antigens detected by Monoclonal Antibodies. (1985). Reinherz, E. L., et al. Eds. Springer-Verlag; Leukocyte Typing III. (1987). White Cell Differentiation Antigens, A. J. McMichael et al., Eds., Oxford University Press.; Leukocyte Typing IV. (1989). White Cell Differentiation Antigens. W. Knapp, et al., Eds., Oxford University Press; Leukocyte Typing V (Vol. I and II). (1995). White Cell Differentiation Antigens. Schlossman, S. F., et al., Eds., Oxford University Press.; and Leukocyte Typing VI. (1997). Kishimoto, T, et al., Eds., Garland Publishing Inc., the contents of all of which are hereby incorporated by reference).

In another embodiment, the immune cell antigen is a chemokine receptor, e.g., a CXC chemokine receptor or a CC chemokine receptor. Non-limiting examples of chemokine receptors include, but are not limited to, CCR1, CCR2A, CCR2B, CCR3, CCR4, CCR5, CCR6, CCR7, CCR8, CCR9, CCR10, CXCR1 (CDwl28a; IL-8 receptor A), CXCR2 (CDwl28b; IL-8 receptor B), CXCR3, and CXCR4.

It should be understood that some of the antigens classified above may be involved in mediating cancer and immune cell responses. For example, CD77, although classified as an immune cell antigen, is associated with Burkitt's lymphoma.

In a preferred embodiment, the T cell antigen is CD3, preferably, the epsilon subunit of CD3. The term “CD3” is art-recognized, and refers to a protein complex, which includes at least five protein chains: an epsilon, delta, and gamma subunit, in association with either a homodimer of two zeta subunits or a heterodimer of a zeta and an eta subunit. As used herein, the term “CD3” refers to any of the CD3 subunits, ε, δ, γ, or eta, alone in combination to form, homodimers or heterodimers. CD3 is typically found on the surface of T cells, and forms a complex with the T cell receptor, (also referred in the art as the “T cell receptor complex”). CD3 is an important component in the propagation of a T cell based immune response, and has been implicated in the pathophysiology of a wide variety of disorders, such as immune disorders or cancers.

Nucleic Acids, Vectors and Host Cells

Another aspect of the invention pertains to isolated nucleic acid, vector and host cell compositions that can be used for recombinant expression of the hybrid antibodies and antigen-binding fragment of the invention. In one embodiment, a first and second isolated nucleic acid comprising a nucleotide sequence encoding heavy and light chain variable regions, respectively, of a hybrid antibody (e.g., a hybrid anti-CD3 antibody), or antigen fragment thereof, are provided. Preferably, the heavy chain variable region is a chimeric heavy chain variable region, e.g., a chimeric heavy chain variable region as described herein, and the light chain variable region is a humanized or CDR-grafted light chain variable region, e.g., a humanized or CDR-grafted light chain variable region as described herein.

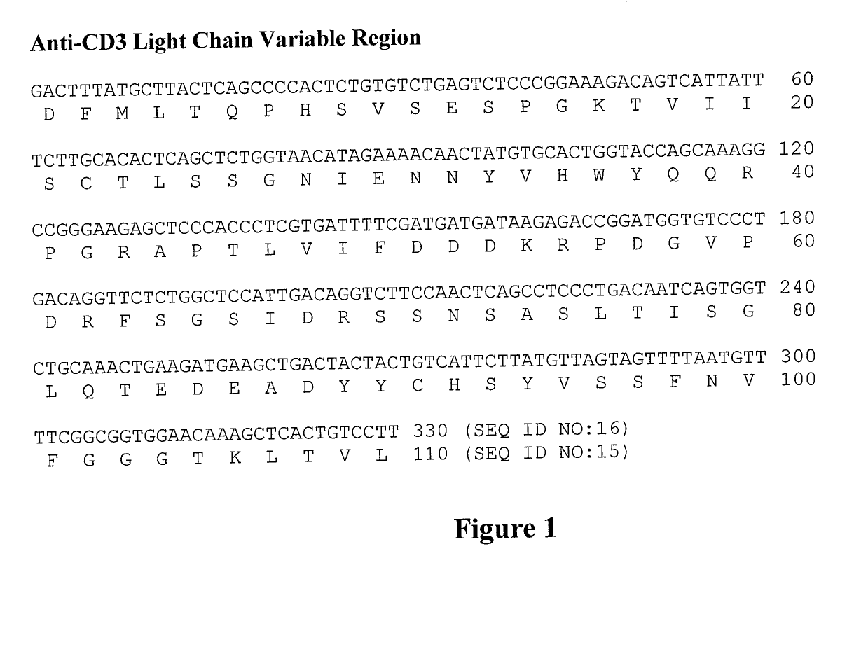

The nucleotide and amino acid sequence of the anti-CD3 hybrid antibody light chain variable region is shown in FIG. 1 (SEQ ID NO:15). The CDR1 domain of the anti-CD3 light chain variable region corresponds to amino acids 23-35 of SEQ ID NO:15 and encompasses nucleotides 67-105 of SEQ ID NO:16; the CDR2 domain of the anti-CD3 light chain variable region corresponds to amino acids 51-57 of SEQ ID NO:15 and encompasses nucleotides 151-171 of SEQ ID NO:16; and the CDR3 domain of the anti-CD3 light chain variable region corresponds to amino acids 92-100 of SEQ ID NO:15 and encompasses nucleotides 274-300 of SEQ ID NO:16.

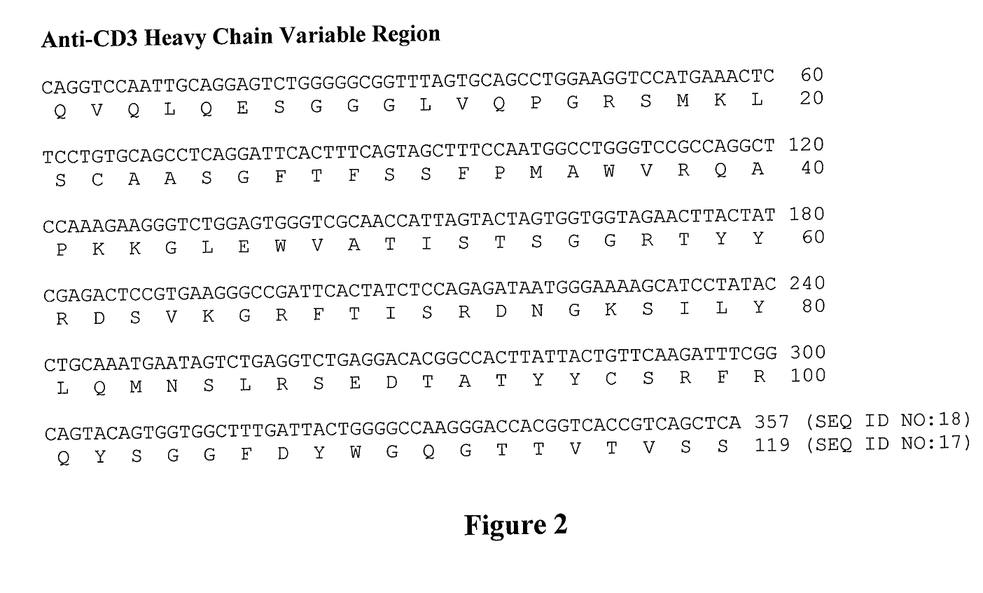

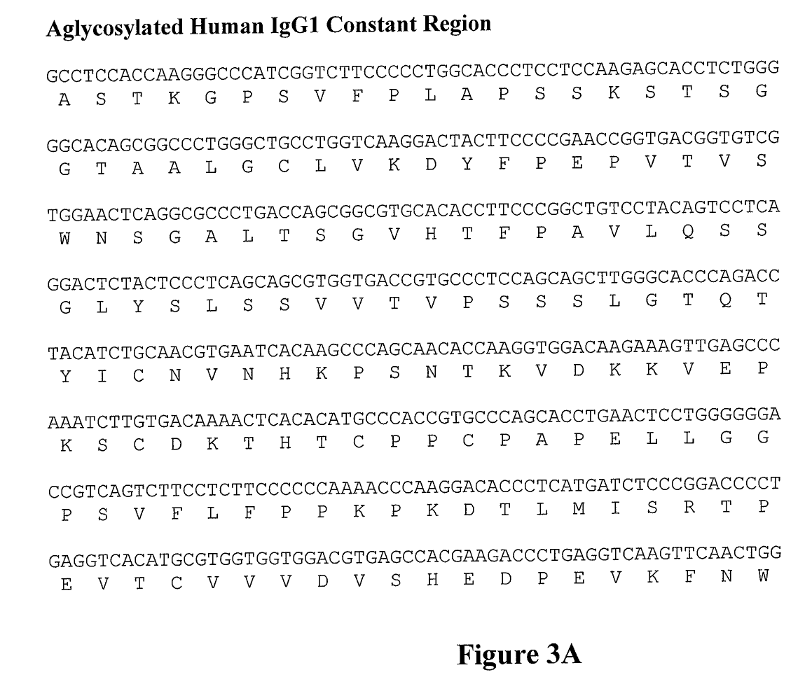

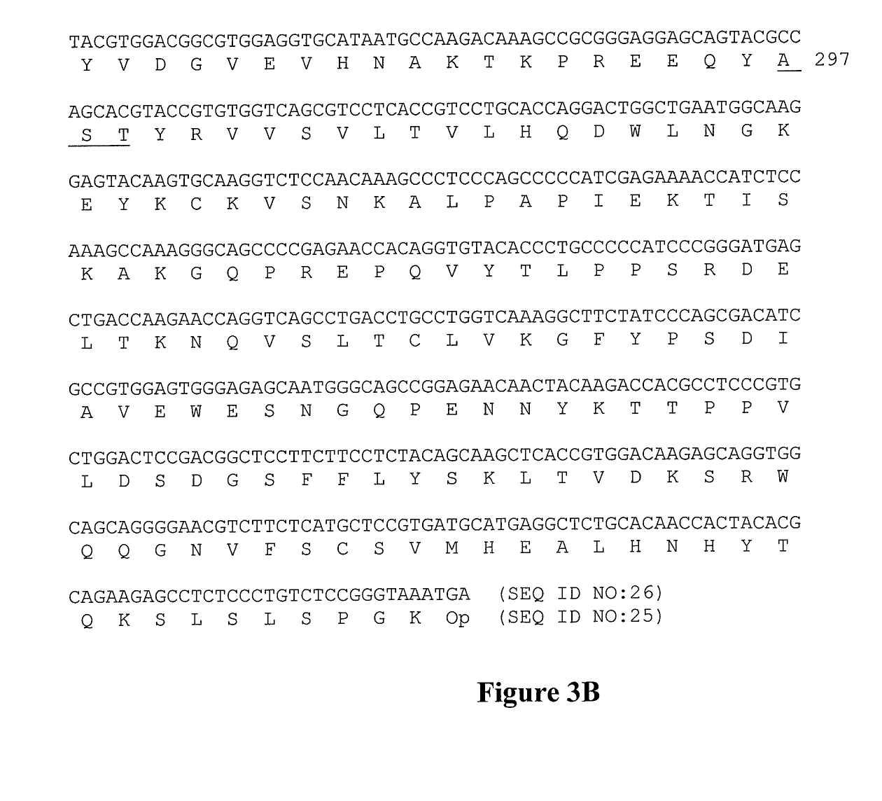

The nucleotide sequence encoding the anti-CD3 hybrid antibody heavy chain variable region is shown in FIG. 2 and SEQ ID NO:18. It will be appreciated by the skilled artisan that nucleotide sequences encoding anti-CD3 hybrid molecules (e.g., a CDR domain, such as a CDR3 domain), can be derived from the nucleotide and amino acid sequences described in the present application using the genetic code and standard molecular biology techniques.

The nucleic acid compositions of the present invention, while often in a native sequence (except for modified restriction sites and the like), from either cDNA, genomic or mixtures may be mutated, thereof in accordance with standard techniques to provide gene sequences. For coding sequences, these mutations, may affect amino acid sequence as desired. In particular, nucleotide sequences substantially identical to or derived from native V, D, J, constant, switches and other such sequences described herein are contemplated (where “derived” indicates that a sequence is identical or modified from another sequence).

In one embodiment, the isolated first nucleic acid comprises an anti-CD3 hybrid antibody heavy chain variable region nucleotide sequence having a nucleotide sequence as shown in FIG. 2 (SEQ ID NO:18), or a sequence at least 85%, 90%, 95%, 99% or higher identical thereto. In another embodiment, the isolated first nucleic acid encodes an anti-CD3 hybrid antibody heavy chain variable region amino acid sequence having an amino acid sequence as shown in FIG. 2 (SEQ ID NO:17), or a sequence at least 85%, 90%, 95%, 99% or higher identical thereto. In another embodiment, the isolated first nucleic acid comprises a nucleotide sequence encoding at least one, preferably two, and most preferably three, CDR's of the heavy chain variable region of the anti-CD3 antibody chosen from the amino acid sequences of SEQ ID NOs:1, 2, and 3, or a CDR sequence which differs by one or two amino acids from the sequences described herein. In yet another embodiment, the isolated first nucleic acid comprises a nucleotide sequence selected from the nucleotide sequences shown in SEQ ID NOs:19, 20, and 21, or a sequence encoding a CDR which differs by one or two amino acids from the sequences described herein. In another embodiment, the isolated first nucleic acid comprises a nucleotide sequence encoding at least one, preferably two, three and most preferably four amino acid sequences from the heavy chain variable framework region of the anti-CD3 hybrid antibody chosen from SEQ ID NO:7, 8, 9, or 10, or a sequence or a sequence at least 85%, 90%, 95%, 99% or higher identical thereto.

In yet another embodiment, the isolated second nucleic acid comprises an anti-CD3 hybrid antibody light chain variable region nucleotide sequence having a sequence as shown in FIG. 1 (SEQ ID NO:16), or a sequence at least 85%, 90%, 95%, 99% or higher identical thereto. In another embodiment, the isolated second nucleic acid encodes an anti-CD3 hybrid antibody light chain variable region amino acid sequence having a sequence as shown in FIG. 1 (SEQ ID NO:15), or a sequence at least 85%, 90%, 95%, 99% or higher identical thereto. In another embodiment, the isolated second nucleic acid comprises a nucleotide sequence encoding at least one, preferably two, and most preferably three, CDR's of the light chain variable region of the anti-CD3 antibody chosen from the amino acid sequences of SEQ ID NOs:4, 5, and 6, or a sequence encoding a CDR which differs by one or two amino acids from the sequences described herein. In yet another embodiment, the isolated second nucleic acid comprises a nucleotide sequence selected from the nucleotide sequences shown in SEQ ID NOs:22, 23, and 24, or a sequence encoding a CDR which differs by one or two amino acids from the sequences described herein. In another embodiment, the isolated second nucleic acid comprises a nucleotide sequence encoding at least one, preferably two, three and most preferably four amino acid sequences from the light chain variable framework region of the anti-CD3 hybrid antibody chosen from SEQ ID NO:11, 12, 13, or 14, or a sequence at least 85%, 90%, 95%, 99% or higher identical thereto.

In a preferred embodiment, isolated first and second nucleic acids have nucleotide sequences encoding a heavy chain and the light chain variable regions of an anti-CD3 antibody having at least one, two, three, four, five and preferably all CDR's chosen from the amino acid sequences of SEQ ID NOs:1, 2, 3, 4, 5, and 6, or sequence encoding a CDR which differs by one or two amino acids from the sequences described herein.

The nucleic acid can encode only the light chain or the heavy chain variable region, or can also encode an antibody light or heavy chain constant region, operatively linked to the corresponding variable region. In one embodiment, the light chain variable region is linked to a constant region chosen from a kappa or a lambda constant region. Preferably, the light chain constant region is from a lambda type (e.g., a human type lambda). In another embodiment, the heavy chain variable region is linked to a heavy chain constant region of an antibody isotype selected from the group consisting of IgG (e.g., IgG1, IgG2, IgG3, IgG4), IgM, IgA1, IgA2, IgD, and IgE. Preferably, the heavy chain constant region is from an IgG (e.g., an IgG1) isotype.

Nucleic acids of the inventor can be chosen for having codons, which are preferred, or non preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

In a preferred embodiment, the nucleic acid differs (e.g., differs by substitution, insertion, or deletion) from that of the sequences provided, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences. The differences are, preferably, differences or changes at nucleotides encoding a non-essential residue(s) or a conservative substitution(s).

In one embodiment, the first and second nucleic acids are linked, e.g., contained in the same vector. In other embodiments, the first and second nucleic acids are unlinked, e.g., contained in the different vector.

In another aspect, the invention features host cells and vectors (e.g., recombinant expression vectors) containing the nucleic acids, e.g., the first and second nucleic acids, of the invention.

The terms “host cell” and “recombinant host cell” are used interchangeably herein. Such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic, e.g., bacterial cells such as E. coli, or eukaryotic, e.g., insect cells, yeast, or preferably mammalian cells (e.g., cultured cell or a cell line). Other suitable host cells are known to those skilled in the art.

Preferred mammalian host cells for expressing the hybrid antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), lymphocytic cell lines, e.g., NS0 myeloma cells and SP2 cells, COS cells, and a cell from a transgenic animal, e.g., e.g., mammary epithelial cell.

In another aspect, the invention features a vector, e.g., a recombinant expression vector. The recombinant expression vectors of the invention can be designed for expression of the hybrid antibodies, or antigen-binding thereof, in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to an antibody encoded therein, usually to the constant region of the recombinant antibody.

In addition to the antibody chain genes, the recombinant expression vectors of the invention carry regulatory sequences that are operatively linked and control the expression of the antibody chain genes in a host cell.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “regulatory sequence” is intended to includes promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from FF-1a promoter and BGH poly A, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the hybrid antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr host cells with methotrexate selection/amplification) and the neo gene (for G418 selection).

In an exemplary system for recombinant expression of a hybrid antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

Methods of Producing Hybrid Antibody Molecules

In another aspect, the invention features a method of providing a hybrid antibody preparation having improved assembly characteristics, said method comprising providing a first nucleic acid, e.g., a first nucleic acid encoding chimeric heavy chain (or a fragment thereof, e.g., the heavy chain variable region) as described herein; providing a second nucleic acid encoding humanized light chain (or a fragment thereof, e.g., the light chain variable region); and introducing said first and second nucleic acids into a host cell, e.g., a host cell as described herein, under conditions that allow expression and assembly of said light and heavy chain variable regions.

A hybrid antibody molecule of the invention can be prepared by recombinant expression of immunoglobulin light and heavy chain genes in a host cell. To express an antibody recombinantly, a host cell is transfected with one or more recombinant expression vectors carrying nucleic acid fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express a hybrid antibody (e.g., an aglycosylated anti-CD3 hybrid antibody), nucleic acid fragments encoding the light and heavy chain variable regions are first obtained. The exemplary embodiment described below provides general methods of producing CD3 specific monoclonal antibodies with chimeric heavy chain and a humanized light chain. Similar protocols can be used to generate antibodies to other predetermined antigens by practicing routine experimentation. The cloning and re-shaping of the V-region gene of the rat antibody YTH 12.5 specific for the human CD3 antigen is performed as described in Bolt, S. et al. (1993) Eur. J. Immunol. 23(2):403-11; Routledge, E. G. et al. (1995) Transplantation 60(8):847-53; U.S. Pat. Nos. 5,585,097; 5,968,509, the contents of all of which are hereby incorporated by reference. YTH 12.5 is a rat hybridoma cell line secreting an IgG2b monoclonal antibody specific for the CD3 antigen complex, but the methodology is applicable to other cells secreting CD3 specific antibodies with the same CDR's.

Briefly, the methodology is based on that of Orlandi et al. (1989) PNAS USA Vol. 86:3833, using the polymerase chain reaction (PCR). The V_{H} or V_{L} gene can be cloned using oligonucleotide primers designed based on published nucleotide sequences, e.g., the oligonucleotide primers described in Routledge, E. G. et al. (1995), supra. To generate a CDR-grafted light chain, the PCR products are ligated into a vector in which site-directed mutagenesis is performed using six oligonucleotide primers. To generate, a chimeric heavy chain variable region, the V_{H} gene can be cloned into a vector. The humanized and chimeric light and heavy chains, respectively, can, optionally, be cloned together with the corresponding constant regions. Constant regions from different species may be used. The cloned light and heavy chains can be cloned into appropriate expression vectors.

An expression vector can be generated in which the chimeric CD3 VH gene may be expressed in conjunction with different immunoglobulin heavy chain constant region genes (Gunning s al. (1987) P. N. A. S. USA 85: 7719-7723). The expression vector may additionally include A 1.65 Kb fragment of DNA carrying the dihydrofolate reductase (dhft) gene and SV 40 expression signals (Page & Sydenham (1991) Biotechnology, 2, 64). The nucleic acid encoding the chimeric heavy chain variable region can be cloned downstream of the B actin promoter or preferably the EF-1a promoter.

The aglycosyl human IgG1 constant region is derived from the wild type G1m (1, 17) gene constant region described by Takahashi et al (1982) Cell 2:671-679. The Glm (1, 17) gene constant region is cloned into a vector where site-directed mutagenesis can be performed (Amersham International PLC kit) to mutate the amino acid residue at position 297 from an asparagine to an alanine residue.

Oligosaccharide at Asn-297 is a characteristic feature of a normal human IgG antibodies (Kabat (1991), Sequence of Proteins of Immunological Interest, US Department of Health Human Services Publication), each of the two heavy chains in the IgG molecules having a single branched chain carbohydrate group which is linked to the amide group of the asparagine residue (Rademacher and Dwek (1984) Prog. Immunol., 2, 95-112). Substitution of asparagine with alanine prevents the glycosylation of the antibody.

Subconfluent monolayers of dhfr-Chinese Hamster Ovary cells can be co-transfected with the vector containing the heavy chain gene and a second vector containing the humanized light chain (Routledge et al. (1991) Eur. J. Immunol.: 21:2717-2725). Alternatively, the heavy and light chain genes can be cloned into a single vector. Prior to transfection, the plasmid DNA(s) can be linearized using the appropriate restriction endonuclease.

Heavy and light chain transfectants can be selected for in xanthine/hypoxanthine free IMDM containing 5%(v/v)dialyzed fetal calf serum.

Hybrid antibodies having different constant regions are described in the art and can be readily produced by one of ordinary skill in the art. For example, the production of the analogous wild type human IgG1-CD3 heavy chain vector has been described elsewhere (Routledge et al. (1991) Eur. J. lmmunol. 21: 2717-2725). Heavy chain expression vectors carrying the non-mutant human IgG2 (Flanagan & Rabbitts (1982) Nature 300: 709-713), IgG3 (Huck et al. (1986) Nucl. Acid. Res. 2: 1779-1789), IgG4 (Flanagan & Rabbitts (1982) supra, Epsilon (Flanagan & Rabbitts (1982) EMBO Journal 1:655-660), and Alpha-2 (Flanagan & Rabbitts (1982) supra.) constant region genes have been decribed in the art. Introduction of these expression vectors, in conjunction with a light chain vector into dhfr-CHO cells as described earlier, produced cell lines secreting CD3 antibody of the above-described isotypes.

A competition assay can be designed to specifically quantitate the concentration of antibody with CD3 antigen binding capacity produced. Human T-cell blasts are incubated with FITC labeled UCHT-1, an antibody which binds to a spatially indistinguishable epitope of the CD3 antigen as the chimeric panel. The concentration of FITC reagent used is previously determined to be half saturating. Unlabeled YTH 12.5 (HPLC purified) can be titrated from a known starting concentration and added to wells containing T-cells and UCHTI-1 FITC. The unlabeled antibody serves as a competitor for the antigen binding site. This is detected as decrease in the mean fluorescence seen when the cells are studied using FACS analysis.

Once nucleic acids encoding the hybrid antibodies of the invention are obtained, as described above, these nucleic acid fragments can be further manipulated by standard recombinant DNA techniques, for example, to convert the variable region genes to full-length antibody chain genes, to Fab fragment genes or to a scFv gene. In these manipulations, a VL- or VH-encoding nucleic acid fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another nucleic acid molecule encoding heavy chain constant regions (CH1, CH2 and CH3). The sequences of heavy chain constant region genes are known in the art. Preferably, the constant region is human, but constant species from other species, e.g., rodent (e.g., mouse or rat), primate, camel, rabbit can also be used. Constant regions from these species are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 constant region. For a Fab fragment heavy chain gene, the VH-encoding DNA can be operatively linked to another DNA molecule encoding only the heavy chain CH1 constant region.

To express the hybrid antibodies, or antibody binding fragments of the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the hybrid antibody light or heavy chain sequences, the expression vector may already carry antibody constant region sequences. For example, one approach to converting the VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immnunoglobulin signal peptide or a heterologous signal peptide (i.e., a signal peptide from a non-immunoglobulin protein).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to a predetermined antigen, e.g., CD3. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the predetermined antigen, e.g., CD3 by crosslinking an antibody of the invention to a second antibody by standard chemical crosslinking methods.

Still further the invention provides a method of producing a recombinant hybrid antibody of the invention by culturing a host cell of the invention in a suitable culture medium until a recombinant hybrid antibody of the invention is synthesized. The method can further comprise isolating the recombinant hybrid antibody from the culture medium.

Pharmaceutical Compositions

In another aspect, the present invention provides compositions, e.g., pharmaceutically acceptable compositions, which include a hybrid antibody molecule described herein, formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., hybrid antibody molecule may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Phann. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The hybrid antibodies and antibody-fragments of the present invention can be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Phannacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the hybrid antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the hybrid antibody or antibody fragment is outweighed by the therapeutically beneficial effects. A “therapeutically effective dosage” preferably inhibits a measurable parameter, e.g., tumor growth by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Combination Therapy

In one embodiment, the compositions of the invention, e.g., the pharmaceutical compositions, are administered in combination therapy, i.e., combined with other agents, e.g., therapeutic agents, that are useful for treating disorders, such as cancer or T cell-mediated disorders. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment. For example, the combination therapy can include a composition of the present invention coformulated with, and/or coadministered with, one or more additional therapeutic agents, e.g., one or more anti-cancer agents, cytotoxic or cytostatic agents and/or immunosuppressants. For example, the hybrid antibodies of the invention or antibody binding fragments thereof may be coformulated with, and/or coadministered with, one or more additional antibodies that bind other targets (e.g., antibodies that bind other cytokines or that bind cell surface molecules), one or more cytokines, or immunosuppressants, e.g., cyclosporin A or FK506. Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies.

The terms “cytotoxic agent” and “cytostatic agent” and “anti-tumor agent” are used interchangeably herein and refer to agents that have the property of inhibiting the growth or proliferation (e.g., a cytostatic agent), or inducing the killing, of hyperproliferative cells, e.g., an aberrant cancer cell or a T cell. In cancer therapeutic embodiment, the term “cytotoxic agent” is used interchangeably with the terms “anti-cancer” or “anti-tumor” to mean an agent, which inhibits the development or progression of a neoplasm, particularly a solid tumor, a soft tissue tumor, or a metastatic lesion.

Nonlimiting examples of anti-cancer agents include, e.g., antimicrotubule agents, topoisomerase inhibitors, antimetabolites, mitotic inhibitors, alkylating agents, intercalating agents, agents capable of interfering with a signal transduction pathway, agents that promotes apoptosis and radiation. Examples of the particular classes of anti-cancer agents are provided in detail as follows: antitubulin/antimicrotubule, e.g., paclitaxel, vincristine, vinblastine, vindesine, vinorelbin, taxotere; topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5-fluorouridine, FUDR, tiazofurin, N-[5-[N-(3,4-dihydro-2-methyl-4-oxoquinazolin-6-ylmethyl)-N-methylamino]-2-thenoyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol; agents acting via other mechanisms of action, e.g., dihydrolenperone, spiromustine, and desipeptide; biological response modifiers, e.g., to enhance anti-tumor responses, such as interferon; apoptotic agents, such as actinomycin D; and anti-hormones, for example anti-estrogens such as tamoxifen or, for example antiandrogens such as 4′-cyano-3-(4-fluorophenylsulphonyl)-2-hydroxy-2-methyl -3′-(trifluoromethyl) propionanilide.

Particular combination of cytotoxic agents can be used depending on the condition to be treated. For example, when treating leukemias, in addition to radiation, the following drugs, usually in combinations with each other, are often used: vincristine, prednisone, methotrexate, mercaptopurine, cyclophosphamide, and cytarabine. In chronic leukemia, for example, busulfan, melphalan, and chlorambucil can be used in combination. All of the conventional anti-cancer drugs are highly toxic and tend to make patients quite ill while undergoing treatment. Vigorous therapy is based on the premise that unless every leukemic cell is destroyed, the residual cells will multiply and cause a relapse.

The hybrid antibody molecules of the invention can be used in combination with other therapeutic agents that inhibit the activity of immune cells to be used to treat immune or hematopoietic cell disorders or conditions, including, but not limited to, transplant rejection, multiple sclerosis, rheumatoid arthritis, inflammatory bowel disease, among others. The use of the antibodies, or antibody fragments, of the invention in combination with other therapeutic agents is discussed in further detail below.

In one embodiment, the hybrid antibody molecules bind to human CD3. The CD3 antigen complex is involved in the process of T cell activation in response to antigen recognition by the T cell receptors. CD3 monoclonal antibodies, including aglycosylated humanized anti-CD3 antibodies, are known in the art to be effective immunosuppressants for treating or preventing a number of autoimmune and/or transplant rejections conditions (Bolt, S. et al. (1993) Eur. J. Immunol. 23(2):403-1 1; Routledge, E. G. et al. (1995) Transplantation 60(8):847-53; U.S. Pat. Nos. 5,585,097; 5968509, the contents of all of which are hereby incorporated by reference).

Accordingly, a hybrid antibody molecule of the invention can be used in combination with one or more antibodies directed at other targets involved in regulating immune responses, e.g., transplant rejection or graft-v-host disease. Non-limiting examples of agents for treating or preventing immune responses with which a hybrid antibody, or antibody portion, of the invention can be combined include the following: antibodies against cell surface molecules, including but not limited to CD25 (interleukin-2 receptor-α), CD11a (LFA-1), CD54 (ICAM-1), CD4, CD45, CD28/CTLA4, CD80 (B7-1) and/or CD86 (B7-2). In yet another embodiment, a hybrid antibody or antibody portion of the invention is used in combination with one or more general immunosuppressive agents, such as cyclosporin A or FK506.

Non-limiting examples of agents for treating or preventing rheumatoid arthritis with which a hybrid antibody, or antibody portion, of the invention can be combined include the following: non-steroidal anti-inflammatory drug(s) (NSAIDs); cytokine suppressive anti-inflammatory drug(s) (CSAIDs); CDP-571/BAY-10-3356 (humanized anti-TNFα antibody; Celltech/Bayer); cA2 (chimeric anti-TNFα antibody; Centocor); 75 kdTNFR-IgG (75 kD TNF receptor-IgG fusion protein; Immunex; see e.g., Arthritis & Rheumatism (1994) Vol. 37, S295; J. Invest. Med. (1996) Vol. 44, 235A); 55 kdTNFR-IgG (55 kD TNF receptor-IgG fusion protein; Hoffmann-LaRoche); IDEC-CE9.1/SB 210396 (non-depleting primatized anti-CD4 antibody; IDEC/SmithKline; see e.g., Arthritis & Rheumatism (1995) Vol. 38, S185); DAB 486-IL-2 and/or DAB 389-IL-2 (IL-2 fusion proteins; Seragen; see e.g., Arthritis & Rheumatism (1993) Vol. 36, 1223); Anti-Tac (humanized anti-IL-2R; Protein Design Labs/Roche); IL-4 (anti-inflammatory cytokine; DNAX/Schering); IL-10 (SCH 52000; recombinant IL-10, anti-inflammatory cytokine; DNAX/Schering); IL-4; IL-10 and/or IL-4 agonists (e.g., agonist antibodies); IL-1RA (IL-1 receptor antagonist; Synergen/Amgen); TNF-bp/s-TNFR (soluble TNF binding protein; see e.g., Arthritis & Rheumatism (1996) Vol. 39, No. 9 (supplement), S284; Amer. J. Physiol.—Heart and Circulatory Physiology (1995) Vol. 268, pp. 37-42); R973401 (phosphodiesterase Type IV inhibitor; see e.g., Arthritis & Rheumatism (1996) Vol. 39, No. 9 (supplement), S282); MK-966 (COX-2 Inhibitor; see e.g., Arthritis & Rheumatism (1996) Vol. 39, No. 9 (supplement), S81); Iloprost (see e.g., Arthritis & Rheumatism (1996) Vol. 39, No. 9 (supplement), S82); methotrexate; thalidomide (see e.g., Arthritis & Rheumatism (1996) Vol. 39, No. 9 (supplement), S282) and thalidomide-related drugs (e.g., Celgen); leflunomide (anti-inflammatory and cytokine inhibitor; see e.g., Arthritis & Rheumatism (1996) Vol. 39, No. 9 (supplement), S131; Inflammation Research (1996) Vol. 45, pp. 103-107); tranexamic acid (inhibitor of plasminogen activation; see e.g., Arthritis & Rheumatism (1996) Vol. 39, No. 9 (supplement), S284); T-614 (cytokine inhibitor; see e.g., Arthritis & Rheumatism (1996) Vol. 39, No. 9 (supplement), S282); prostaglandin E1 (see e.g., Arthritis & Rheumatism (1996) Vol. 39, No. 9 (supplement), S282); Tenidap (non-steroidal anti-inflammatory drug; see e.g., Arthritis & Rheumatism (1996) Vol. 39 No. 9 (supplement), S280); Naproxen (non-steroidal anti-inflammatory drug; see e.g., Neuro Report (1996) Vol. 7, pp. 1209-1213); Meloxicam (non-steroidal anti-inflammatory drug); Ibuprofen (non-steroidal anti-inflammatory drug); Piroxicam (non-steroidal anti-inflammatory drug); Diclofenac (non-steroidal anti-inflammatory drug); Indomethacin (non-steroidal anti-inflammatory drug); Sulfasalazine (see e.g., Arthritis & Rheumatism (1996) Vol. 39, No. 9 (supplement), S281); Azathioprine (see e.g., Arthritis & Rheumatism (1996) Vol. 39, No. 9 (supplement), S28 1); ICE inhibitor (inhibitor of the enzyme interleukin-1 converting enzyme); zap-70 and/or Ick inhibitor (inhibitor of the tyrosine kinase zap-70 or Ick); VEGF inhibitor and/or VEGF-R inhibitor (inhibitos of vascular endothelial cell growth factor or vascular endothelial cell growth factor receptor; inhibitors of angiogenesis); corticosteroid anti-inflammatory drugs (e.g., SB203580); TNF-convertase inhibitors; anti-IL-12 antibodies; interleukin-11 (see e.g., Arthritis & Rheumatism (1996) Vol. 39, No. 9 (supplement), S296); interleukin-13 (see e.g., Arthritis & Rheumatism (1996) Vol. 39, No. 9 (supplement), S308); interleukin-17 inhibitors (see e.g., Arthritis & Rheumatism (1996) Vol. 39, No. 9 (supplement), S120); gold; penicillamine; chloroquine; hydroxychloroquine; chlorambucil; cyclophosphamide; cyclosporine; total lymphoid irradiation; anti-thymocyte globulin; anti-CD4 antibodies; CD5-toxins; orally-administered peptides and collagen; lobenzarit disodium; Cytokine Regulating Agents (CRAS) HP228 and HP466 (Houghten Pharmaceuticals, Inc.); ICAM-1 antisense phosphorothioate oligodeoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline; anti-IL2R antibodies; marine and botanical lipids (fish and plant seed fatty acids; see e.g., DeLuca et al. (1995) Rheum. Dis. Clin. North Am. 21:759-777); auranofin; phenylbutazone; meclofenamic acid; flufenamic acid; intravenous immune globulin; zileuton; mycophenolic acid (RS-61443); tacrolimus (FK-506); sirolimus (rapamycin); amiprilose (therafectin); cladribine (2-chlorodeoxyadenosine); and azaribine.

Nonlimiting examples of agents for treating or preventing inflammatory bowel disease with which a hybrid antibody, or antibody portion, of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies; growth factors; elastase inhibitors; pyridinyl-imidazole compounds; CDP-571/BAY-10-3356 (humanized anti-TNFα antibody; Celltech/Bayer); cA2 (chimeric anti-TNFα antibody; Centocor); 75 kdTNFR-IgG (75 kD TNF receptor-IgG fusion protein; Immunex; see e.g., Arthritis & Rheumatism (1994) Vol. 37, S295; J. Invest. Med. (1996) Vol. 44, 235A); 55 kdTNFR-IgG (55 kD TNF receptor-IgG fusion protein; Hoffmann-LaRoche); interleukin-10 (SCH 52000; Schering Plough); IL-4; IL-10 and/or IL-4 agonists (e.g., agonist antibodies); interleukin-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of Platelet Activating Factor (PAF); ciprofloxacin; and lignocaine.

Nonlimiting examples of agents for treating or preventing multiple sclerosis with which an hybrid antibody, or antibody portion, of the invention can be combined include the following: corticosteroids; prednisolone; methylprednisolone; azathioprine; cyclophosphamide; cyclosporine; methotrexate; 4-aminopyridine; tizanidine; interferon-α1a (Avonex™; Biogen); interferon-1b (Betaseron™; Chiron/Berlex); Copolymer 1 (Cop-1; Copaxone™; Teva Pharmaceutical Industries, Inc.); hyperbaric oxygen; intravenous immunoglobulin; clabribine; CDP-571/BAY-10-3356 (humanized anti-TNFα antibody; Celltech/Bayer); cA2 (chimeric anti-TNFα antibody; Centocor); 75 kdTNFR-IgG (75 kD TNF receptor-IgG fusion protein; Immunex; see e.g., Arthritis & Rheumatism (1994) Vol. 37, S295; J. Invest. Med. (1996) Vol. 44, 235A); 55 kdTNFR-IgG (55 kD TNF receptor-IgG fusion protein; Hoffmann-LaRoche); IL-10; IL-4; and IL-10 and/or IL-4 agonists (e.g., agonist antibodies).

Another aspect of the present invention accordingly relates to kits for carrying out the combined administration of the hybrid antibody molecules with other therapeutic compounds. In one embodiment, the kit comprises a hybrid antibody formulated in a pharmaceutical carrier, and at least one cytotoxic agent, formulated as appropriate, in one or more separate pharmaceutical preparations.

Uses of the Invention

The hybrid antibodies molecules have in vitro and in vivo diagnostic and therapeutic utilities. For example, these antibodies can be administered to cells in culture, e.g. in vitro or ex vivo, or in a subject, e.g., in vivo, to treat or diagnose a variety of disorders. As used herein, the term “subject” is intended to include human and non-human animals. Preferred human animals include a human patient having a disorder characterized by abnormal functioning of a cell expressing a predetermined antigen, e.g., a cancer cell or an immune cell (e.g., a T cell, e.g., a CD3-expressing cell). The term “non-human animals” of the invention includes all vertebrates, e.g., mammals and non-mammals, such as non-human primates, sheep, dog, cow, chickens, amphibians, reptiles, etc.

Accordingly, in one aspect, the present invention provides a diagnostic method for detecting the presence of an antigen recognized by a hybrid antibody, or an antigen-binding fragment thereof, in vitro (e.g., a biological sample, such as serum, plasma, tissue, biopsy) or in vivo (e.g., in vivo imaging in a subject). The method includes: (i) contacting the sample with , or administering to the subject, the hybrid antibody molecule; (ii) contacting a control sample (e.g., a control biological sample, such as serum, plasma, tissue, biopsy) or a control subject)); and (iii) detecting formation of a complex between the hybrid antibody (or fragment thereof), and the sample or subject, or the control sample or subject, wherein a statistically significant change in the formation of the complex in the sample or subject relative to the control sample or subject is indicative of the presence of the antigen in the sample.

Preferably, the hybrid antibody molecule is directly or indirectly labeled with a detectable substance to facilitate detection of the bound or unbound antibody. Suitable detectable substances include various enzymes, prosthetic groups, fluorescent materials, luminescent materials and radioactive materials, as described above. Examples of suitable enzymes include horseradish peroxidase, alkaline phosphatase, β-galactosidase, or acetylcholinesterase; examples of suitable prosthetic group complexes include streptavidintbiotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

Complex formation between the hybrid antibody molecule and the antigen can be detected by measuring or visualizing either the hybrid antibody (or antibody fragment) bound to the antigen or unbound antibody (or antibody fragment). Conventional detection assays can be used, e.g., an enzyme linked immunosorbent assays (ELISA), an radioimmunoassay (RIA) or tissue immunohistochemistry.

Alternative to labeling the hybrid antibody molecule, a predetermined antigen can be assayed in a sample by a competition immunoassay utilizing standards labeled with a detectable substance and an unlabeled anti-antigen antibody. In this assay, the biological sample, the labeled standards and the hybrid antibody molecule are combined and the amount of labeled standard bound to the unlabeled antibody is determined. The amount of antigen in the sample is inversely proportional to the amount of labeled standard bound to the hybrid antibody. A hybrid antibody molecule can also be used to detect antigens from species other than particular species of the antigen. For example, if the antigen recognized by the hybrid antibody molecule is of human origin, antigens from different species, such as primates (e.g., chimpanzee, baboon, marmoset, cynomolgus and rhesus), pig and mouse, can also be detected.

In still another embodiment, the invention provides a method for detecting the presence of an antigen-(e.g., a CD3)-expressing cell in vivo. The method comprises (i) administering to a subject a hybrid antibody molecule, conjugated to a detectable marker; (ii) exposing the subject to a means for detecting said detectable marker to the antigen-expressing cells. Protocols for in vivo diagnostic assays are provided in PCT/US88/01941, EP 0 365 997 and U.S. Pat. No. 4,954,617.

The hybrid antibody molecules (e.g., the anti-CD3 hybrid antibodies) of the invention are capable of binding to their corresponding antigen, e.g., CD3, with high affinity and specificity in vitro. The modified constant region of the hybrid anti-CD3 antibodies described herein does not significantly trigger a first dose response. Accordingly, the hybrid antibody molecules can be used to modulate (e.g., inhibit or reduce) the activity of the antigen with which it reacts (e.g., e.g., in a cell culture containing the antigen, or in a subject).

In one embodiment, the invention provides a method for inhibiting the activity, or expression, of a predetermined antigen, comprising contacting the antigen with a hybrid antibody molecule, such that the antigen activity is inhibited. In one embodiment, the antigen is CD3, e.g., human CD3. For example, a hybrid antibody molecule can be added to a subject, alone or in combination with a therapeutic agent, to inhibit the activity of a cell expressing the antigen (e.g., CD3-expressing cell, e.g., a T cell).

The invention provides methods for treating, or preventing, in a subject, a disease or a disorder involving aberrant activity of a cell expressing an antigen recognized by the hybrid antibody molecules of the present invention. The method comprises administering to the subject a hybrid antibody molecule such that aberrant cell activity in the subject is inhibited. In one embodiment, the antigen is CD3, preferably human CD3, and the subject is a human subject. Alternatively, the subject can be a mammal expressing an antigen with which a hybrid antibody of the invention cross-reacts. A hybrid antibody molecule of the invention can be administered to a human subject for therapeutic purposes (discussed further below). Moreover, a hybrid antibody molecule of the invention (e.g., the anti-CD3 antibody) can be administered to a non-human mammal expressing the antigen with which the hybrid antibody cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

The antigen recognized by the hybrid antibodies or antigen-binding fragments thereof of the invention can be, e.g., a cancer or an immune cell antigen. Accordingly, the hybrid antibodies, or antigen binding fragments thereof, of the invention can be used to treat, prevent, and/or diagnose disorders, such as cancers and immune cell disorders, e.g., T cell disorders.

As used herein, the terms “cancer”, “hyperproliferative”, “malignant”, and “neoplastic” are used interchangeably, and refer to those cells an abnormal state or condition characterized by rapid proliferation or neoplasm. The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be either benign, premalignant or malignant.

The subject method can be useful in treating malignancies of the various organ systems, such as those affecting lung, breast, lymphoid, gastrointestinal (e.g., colon), and genitourinary tract (e.g., prostate), pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

The subject method can also be used to inhibit the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97). Lymphoid malignancies which may be treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated by the treatment method of the present invention include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin's disease.

As used herein, the terms “leukemia” or “leukemic cancer” refers to all cancers or neoplasias of the hematopoietic and immune systems (blood and lymphatic system). These terms refer to a progressive, malignant disease of the blood-forming organs, marked by distorted proliferation and development of leukocytes and their precursors in the blood and bone marrow. The acute and chronic leukemias, together with the other types of tumors of the blood, bone marrow cells (myelomas), and lymph tissue (lymphomas), cause about 10% of all cancer deaths and about 50% of all cancer deaths in children and adults less than 30 years old. Chronic myelogenous leukemia (CML), also known as chronic granulocytic leukemia (CGL), is a neoplastic disorder of the hematopoietic stem cell.

The subject method can also be used to modulate (e.g., inhibit) the activity, e.g., proliferation, differentiation, survival) of an immune or hematopoietic cell (e.g., a cell of myeloid, lymphoid, erythroid lineages, or precursor cells thereof), and, thus, can be used to treat or prevent a variety of immune disorders. Non-limiting examples of the disorders that can be treated or prevented include, but are not limited to, transplant rejection, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, and allergy such as, atopic allergy.

In one embodiment, the invention provides a method for inhibiting an antigen, e.g., CD3, activity or expression, in a subject, suffering from a disorder in which the antigen, e.g., CD3, activity or expression is detrimental. CD3 is an important component of the T cell receptor and is required for a T cell based immune response. Therefore, inhibition of CD3 has been implicated in the control of a wide variety of immunological disorders, e.g., transplant rejection or autoimmune disorders, and cancers. The anti-tumor antibodies described herein have therapeutic potential in the treatment of cancers.

Methods of administering hybrid antibody molecules are described above. Suitable dosages of the molecules used will depend on the age and weight of the subject and the particular drug used. The hybrid antibody molecules can be used as competitive agents for ligand binding to inhibit, reduce an undesirable interaction. For example, anti-CD3 hybrid antibody can be used to inhibit T cell activation. The hybrid antibody molecules of the invention can also be used directly in vivo to eliminate antigen-expressing cells via natural complement or ADCC mechanisms. The molecules can be coupled to radionuclides, as described in Goldenberg, D. M. et al. (1981) Cancer Res. 41: 4354-4360, and in EP 0365 997. The bispecific or multispecific molecules of the invention can also be coupled to another agent, e.g., an anti-cancer or anti-T cell agent described above. The coupling can be covalently or non-covalently (e.g., agent-containing liposomes that are directed to a desired antigen-expressing cell via the hybrid antibody or fragment thereof).

Therapy with the hybrid antibodies or fragments thereof can be performed in conjunction with other techniques for removal of targeted cells. For example, anti-tumor therapy using hybrid antibodies or fragments thereof of the invention can be used in conjunction with surgery, chemotherapy or radiotherapy.

Hybrid antibody molecules of the invention, which have complement binding sites, such as portions from IgG1, -2, or -3 or IgM which bind complement can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with a hybrid antibodies or fragments thereof of the invention can be improved by binding of complement proteins. In another embodiment target cells coated with the hybrid antibodies or fragments thereof can also be lysed by complement.

Also within the scope of the invention are kits comprising the hybrid antibody molecules of the invention and instructions for use. The kit can further contain a least one additional reagent, such as a therapeutic agent, e.g., a therapeutic agent as described herein, or one or more additional hybrid antibody molecules of the invention.

The contents of all references, pending patent applications and published patents, cited throughout this application are hereby expressly incorporated by reference.

Equivalents

Those skilled in the art will recognize, or be able to ascertain using no more than routine experimentation, many equivalents of the specific embodiments of the invention described herein. Such equivalents are intended to be encompassed by the following claims.

We claim:

1. A hybrid antibody molecule, comprising:
at least one light chain variable region comprising all three complementarity determining region (CDR's) from a donor immunoglobulin and a light chain variable region framework from an acceptor immunoglobulin; and
at least one heavy chain variable region selected from the group consisting of:
(a) a heavy chain variable region which is at least 95% identical to the heavy chain variable region of the donor immunoglobulin; and
(b) a heavy chain variable region from the donor immunoglobulin.

2. The hybrid antibody molecule of claim 1, wherein the acceptor immunoglobulin is a human immunoglobulin.

3. The hybrid antibody molecule of claim 1, wherein the heavy chain variable region is a fully rodent immunoglobulin sequence.

4. The hybrid antibody molecule of claim 1, wherein the light chain variable region is a humanized or a CDR-grafted immunoglobulin chain.

5. The hybrid antibody molecule of claim 1, wherein the heavy chain variable region is a chimeric chain.

6. The hybrid antibody molecule of claim 1, which binds to an antigen with an affinity constant between 10⁸ M⁻¹ and 10¹⁰ M⁻¹.

7. The hybrid antibody molecule of claim 1, which comprises two heavy chains and two light chains.

8. The hybrid antibody molecule of claim 1, which comprises a constant region selected from the group consisting of kappa, lambda, alpha, gamma, delta, epsilon and mu constant region genes.

9. The hybrid antibody molecule of claim 8, wherein the heavy chain or the light chain constant region is from human origin.

10. The hybrid antibody molecule of claim 8, wherein the heavy chain constant region is of a human isotype selected from the group consisting of IgG1, IgG2, IgG3, IgG4, IgM, IgA1, IgA2, IgAsec, IgD, and IgE.

11. The hybrid antibody molecule of claim 8, wherein the heavy chain constant region is of human isotype IgG1.

12. The hybrid antibody of claim 1, which binds to an immune cell antigen.

13. The hybrid antibody molecule of claim 12, wherein the immune cell antigen is selected from the group consisting of CD1, CD2, CD3, CD4, CD5, CD8, CD18, CD20, CD23, CD40L, CD80, and CD86.

14. The hybrid antibody molecule of claim 12, wherein the immune cell antigen is a chemokine receptor selected from the group consisting of a CXC chemokine receptor and a CC chemokine receptor.

15. The hybrid antibody molecule of claim 14, wherein the CC chemokine receptor is selected from the group consisting of a CCR1, CCR2A, CCR2B, CCR3, CCR4, CCR5, CCR6, CCR7, CCR8 and CCR9.

16. The hybrid antibody molecule of claim 1, which binds to a tumor antigen.

17. The hybrid antibody molecule of claim 16, wherein the tumor antigen is selected from the group consisting of EGFR, Her2/neu, HELP, GCC, PSMA, PSA, CD66-c, prostasin, TMPRSS3, TADG 12 and TADG 15.

18. A hybrid antibody molecule, comprising at least one humanized or CDR-grafted light chain variable region and at least one chimeric heavy chain.

19. An anti-CD3 hybrid antibody molecule, comprising:
at least one light chain variable region comprising all three CDR's from a first donor species immunoglobulin and a light chain variable region framework from an acceptor immunoglobulin; and
at least one heavy chain variable region selected from the group consisting of:
(a) a heavy chain variable region at least 95% identical to the heavy chain variable region of the donor immunoglobulin; and
(b) a heavy chain variable region from the donor immunoglobulin.

20. The anti-CD3 antibody molecule of claim 19, wherein the donor is a rat or a mouse.

21. The anti-CD3 antibody molecule of claim 19, wherein the heavy chain variable region has at least one CDR selected from the group of amino acid sequences of SEQ ID NOs:1, 2, and 3.

22. The anti-CD3 antibody molecule of claim 20, wherein the light chain variable region has at least one CDR selected from the group of amino acid sequences of SEQ ID NOs:4, 5, and 6.

23. The anti-CD3 antibody molecule of claim 19, wherein the heavy chain variable framework region has at least one amino acid sequence selected from the group of consisting of SEQ ID NOs:7, 8, 9, and 10.

24. The anti-CD3 antibody molecule of claim 19, wherein the light chain variable framework region has at least one amino acid sequence selected from the group of consisting of SEQ ID NOs:11, 12, 13, and 14.

25. The anti-CD3 antibody molecule of claim 19, wherein the heavy chain variable region has the amino acid sequence shown in SEQ ID NO:17.

26. The anti-CD3 antibody molecule of claim 19, wherein the light chain variable region has the amino acid sequence shown in SEQ ID NO:15.

27. The anti-CD3 antibody molecule of claim 19, wherein the light chain variable region is linked to a human type lambda constant region.

28. The anti-CD3 antibody molecule of claim 19, wherein the heavy chain variable region is linked to a heavy chain constant region of an IgG1 isotype.

29. The anti-CD3 antibody molecule of claim 19, wherein the heavy chain constant region is aglycosylated.

30. The anti-CD3 antibody molecule of claim 29, wherein the asparagine residue at position 297 of the constant region is modified.

31. A pharmaceutical composition comprising the hybrid antibody molecule of either claim 1 or 19, and a pharmaceutically acceptable carrier.

32. A first and second nucleic acid sequences encoding heavy and light chain variable regions, respectively, of a hybrid antibody molecule,
wherein the heavy chain variable region from a donor immunoglobulin; and
wherein a light chain variable region comprises all three CDR's from a donor immunoglobulin and a light chain variable region framework from an acceptor immunoglobulin.

33. A method of providing a modified antibody preparation having improved assembly characteristics, comprising:
providing a first nucleic acid encoding a heavy chain variable region selected from the group consisting of:
(a) a heavy chain variable region at least 95% identical to the heavy chain variable region of a donor immunoglobulin; and
(b) a heavy chain variable region from the donor immunoglobulin;
providing a second nucleic acid encoding a light chain variable region comprising all three CDR's from a donor immunoglobulin and a light chain variable region framework from an acceptor immunoglobulin; and
introducing said first and second nucleic acids into a host cell under conditions that allow expression and assembly of said light and heavy chain variable regions.

34. The method of claim 33, wherein the first and second nucleic acids are linked or unlinked.

35. The method of claim 33, wherein the host cell is a mammalian cell.

36. The method of claim 35, wherein the mammalian cell is selected from the group consisting of a lymphocytic cell line, CHO, COS cells, and a cell from a transgenic animal.

37. A method of modulating the activity of an immune or a cancer cell, comprising contacting the cell with the hybrid antibody molecule of either claim 1 or 19, such that the activity of the cell is modulated.

38. A method of treating or preventing an immune or a cancer disorder, comprising administering to a subject the hybrid antibody molecule of either claim 1 or 19, in an amount effective to treat or prevent the disease.

39. A method for detecting the presence of an antigen recognized by a hybrid antibody molecule in a sample, comprising:
(i) contacting a sample or a control sample with a labeled hybrid antibody molecule of either claim 1 or 19, under conditions that allow interaction of the antibody and the antigen to occur, and
(ii) detecting formation of a complex,
wherein a statistically significant change in the formation of the complex between the labeled hybrid antibody and the antigen with respect to a control sample is indicative the presence of the antigen in the sample.
